SEQUENCE LISTING (9 sequences)

SEQ ID NO: 1 - DNA, 20 bp, Artificial Sequence
  synthetic
ctccatagaggagttgttgc

SEQ ID NO: 2 - DNA, 20 bp, Artificial Sequence
  synthetic
tcacgcttttctaccacttc

SEQ ID NO: 3 - protein, 318 aa, Artificial Sequence
  synthetic
MLSESSSFLKGVMLGSIFCALITMLGHIRIGHGNRMHHHEHHHLQAPNKEDILKISEDERMELSKSFRVYCIILVKPKDVSLWAAVKETWTKHCDKAEFFSSENVKVFESINMDTNDMWLMMRKAYKYAFDKYRDQYNWFFLARPTTFAIIENLKYFLLKKDPSQPFYLGHTIKSGDLEYVGMEGGIVLSVESMKRLNSLLNIPEKCPEQGGMIWKISEDKQLAVCLKYAGVFAENAEDADGKDVFNTKSVGLSIKEAMTYHPNQVVEGCCSDMAVTFNGLTPNQMHVMMYGVYRLRAFGHIFNDALVFLPPNGSDND

SEQ ID NO: 4 - protein, 275 aa, Artificial Sequence
  synthetic
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSS

SEQ ID NO: 5 - protein, 251 aa, Artificial Sequence
  synthetic
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSS

SEQ ID NO: 6 - protein, 266 aa, Artificial Sequence
  synthetic
MDFQVQIFSFLLISASVIMSRGVHSQVQLQESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGTTVTVSSGGGGSGGGGSGGGGSDIQLTQSPSSLSASVGDRVTITCSTSSSVSYMHWYQQKPGKAPKLLIYSTSNLASGVPSRFSGSGSGTDFTFTISSLQPEDIATYYCHQWSSYPTFGQGTKVEIKV

SEQ ID NO: 7 - protein, 15 aa, Artificial Sequence
  synthetic
EPKSCDKTHTCPPCP

SEQ ID NO: 8 - protein, 68 aa, Artificial Sequence
  synthetic
FWVLVVVGGVLACYSLLVTVAFIIFWVRSKRSRLLHSDYMNMTPRRPGPTRKHYQPYAPPRDFAAYRS

SEQ ID NO: 9 - protein, 112 aa, Artificial Sequence
  synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2021/0290646 A1
(43) Pub. Date: Sep. 23, 2021

(54) COMBINATION OF A GLYCOSYLATION INHIBITOR WITH ONE CAR CELL THERAPY FOR TREATING CANCER

(71) Applicants: OSPEDALE SAN RAFFAELE S.R.L., Milano (MI) (IT); FONDAZIONE CENTRO SAN RAFFAELE, Milano (MI) (IT)

(72) Inventors: Attilio BONDANZA, Milano (MI) (IT); Monica CASUCCI, Milano (MI) (IT); Beatrice GRECO, Milano (MI) (IT)

(73) Assignees: OSPEDALE SAN RAFFAELE S.R.L., Milano (MI) (IT); FONDAZIONE CENTRO SAN RAFFAELE, Milano (MI) (IT)

(21) Appl. No.: 17/261,928

(22) PCT Filed: Jul. 22, 2019
(86) PCT No.: PCT/EP2019/069712

§ 371 Date: Jan. 21, 2021

(30) Foreign Application Priority Data
Jul. 23, 2018 (EP) 18185006.6
Mar. 8, 2019 (EP) 19161718.2

(51) Int. Cl. A61K 31/7004; A61K 35/17; A61K 39/395; A61K 31/145; A61K 31/437; A61K 31/7008; A61K 31/7072; A61K 31/407; A61K 45/06; A61P 35/00; A61K 38/17; A61P 35/02; C12N 5/0783

(52) U.S. Cl. CPC A61K 31/7004; A61K 2039/545; A61K 39/3955; A61K 31/145; A61K 31/437; A61K 31/7008; A61K 31/7072; A61K 31/407; A61K 45/06; A61P 35/00; A61K 38/1774; A61P 35/02; C12N 5/0636; C12N 2510/00; A61K 35/17

(57) ABSTRACT

The present invention relates to at least one glycosylation inhibitor for use in combination with CAR cell therapy. Preferably, the glycosylation inhibitor improves the therapeutic potential of the CAR cell therapy. The invention also relates to a pharmaceutical composition and to population or subpopulation of CAR cell that has been contacted with at least one glycosylation inhibitor.

### TECHNICAL FIELD

The present invention relates to at least one glycosylation inhibitor for use in combination with an immunotherapy, preferably said at least one glycosylation inhibitor improves therapeutic potential of said immunotherapy. The immunotherapy may be a cell-based immunotherapy, preferably a CAR cell therapy, preferably a CAR-T cell therapy. The invention also relates to pharmaceutical composition and to population or subpopulation of immune cells that have been contacted with at least one glycosylation inhibitor.

### BACKGROUND ART

Chimeric antigen receptors (CARs) are synthetic biology molecules commonly constructed by fusing an antigen-binding moiety derived from a tumour-reactive monoclonal antibody with intracellular signalling domains derived from T lymphocytes. In the last years, different institutions demonstrated impressive and durable clinical responses by infusing CD19 CAR-T cells in patients with refractory B-cell malignancies (Lee D W, Lancet. February 7; 385(9967):517-528; Turtle C J et al., J Clin Invest. 2016 Jun. 1; 126(6):2123-38; Turtle C J et al., Science Transl Med. 2016 Sep. 7; 8(355):355ra116; Schuster S J et al., N Engl J Med. 2017 Dec. 28; 377(26):2545-2554; Maude S L et al., N Engl J Med. 2018 Feb. 1; 378(5):439-448; Park J H et al., N Engl J Med. 2018 Feb. 1; 378(5):449-459), culminating with the recent approval of the first two CAR-T cell therapies to treat paediatric and young adult relapsed/refractory B-cell ALL (Kymriah, Novartis) and adult relapsed/refractory large B-cell lymphomas (Yescarta, Kite Pharma-Gilead Science).

Despite widespread and justified excitement, the successful application of CAR-T cells to other haematological malignancies and, most importantly, solid tumours remain to be demonstrated. To widen the application of CAR-T cells to other disease indications, dissecting the factors determining their efficacy and toxic profiles becomes crucial. It is well recognized that susceptibility of tumour cells to killing by CAR-T cell depends on multiple factors, including expression levels of the target antigen, availability of accessory molecules, CAR affinity and design of the CAR extracellular spacer.

Glycosylation is the enzymatic process that produces glycosidic linkages of saccharides to other saccharides, proteins or lipids. In particular, glycoproteins carry one or more glycans covalently attached to a polypeptide backbone, usually via nitrogen or oxygen linkages, in which case they are known as N-glycans or O-glycans, respectively. Glycosylation is the most complex post-translational modification of proteins and is involved in many physiological events, such as host-pathogen interaction, cell differentiation and trafficking, and intracellular and intercellular signalling. In general, glycosylated proteins carry different types of antigenic epitopes, including oligosaccharide epitopes, glyco-peptidic epitopes and peptidic epitopes (Lisowska E et al., Cell Mol Life Sci. 2002 March; 59(3):445-55). Interestingly, especially in richly glycosylated proteins, peptidic epitopes can be masked by glycans, as reported for the influenza virus hemagglutinin (Munk K et al., Glycobiology. 1992 June; 2(3):233-40) and the human MUC1 protein (Spencer D I et al., Cancer Letters. 1996 Feb. 27; 100(1-2):11-5). Tumour cells display a wide range of glycosylation alterations compared with their non-transformed counterparts. These changes comprise increased branching of N-glycans, higher density of 0-glycans, generation of truncated versions of normal counterparts, and generation of unusual forms of terminal structures with sialic acid and fucose. Changes in oligosaccharide structures of glycoproteins are involved in cancer progression through the deregulation of cell cycle, induction of cell proliferation, promotion of tumour dissemination and angiogenesis, and facilitation of immune evasion (Pinho S S et al., Nat Rev Cancer. 2015 September; 15(9):540-55).

### SUMMARY OF THE INVENTION

The disclosure features, at least in part, compositions and methods of treating disorders such as cancer (e.g., solid or hematopoietic tumors or tumors associated with tumor-associated macrophages) using immune effector cells (e.g., T cells or NK cells) that express a chimeric antigen receptor (CAR) molecule, e.g., a CAR that binds to a tumor antigen, e.g., an antigen expressed on the surface of a solid tumor or tumor associated with tumor-associated macrophages. The compositions include, and the methods include administering, immune effector cells (e.g., T cells or NK cells) expressing a tumor targeting CAR, in combination with an inhibitor of glycosylation. In some embodiments, the combination maintains or has better clinical effectiveness, e.g., against a solid or blood tumor or tumor associated with tumor-associated macrophages, as compared to either therapy alone. Without being bound by theory, it is shown herein that use of an inhibitor of glycosylation (e.g., as described herein) reduces N-glycan branching, increases tumor-induced activation and cytokine release by CAR-expressing cells, e.g., CAR-expressing T cells, increases tumor recognition and killing by CAR-expressing cells, thereby removing a source of inhibition of a function of CAR-expressing cells, e.g., an antitumor or proliferative activity of the CAR-expressing cells. The invention further pertains to the use of engineered cells, e.g., immune effector cells (e.g., T cells or NK cells), that express a CAR molecule that binds to a tumor antigen, e.g., a solid tumor antigen or antigen on a tumor cell associated with tumor-associated macrophages, in combination with an inhibitor of inhibitor of glycosylation to treat a disorder associated with expression of a tumor antigen, e.g., a solid or hematopoietic tumor antigen or antigen on a tumor associated with tumor-associated macrophages (e.g., a cancer).

The adoptive transfer of CAR-T cells demonstrated impressive results against B-cell malignancies but still limited efficacy against solid cancers. Since solid tumors display a wide range of glycosylation alterations, including increased N-glycan branching, it was hypothesized that antigenic epitopes may be masked from CAR-T cell targeting. Accordingly, the inventors observed that CAR-T cells eliminated more efficiently glycosylation-incompetent than glycosylation-competent tumor cells. To exploit this mechanism in order to increase the efficacy of CAR-T cells, they sought to block glycosylation, in particular N-glycosylation in pancreatic adenocarcinoma cells with the glucose/mannose analogue 2-Deoxy-D-glucose (2DG) and focused on CD44v6 as model antigen. Similarly to glycosylation knocked-out cells, treatment with 2DG caused membrane exposure of de-glycosylated antigens, thus sensitizing tumor cells to recognition by CAR-T cells. Notably, 2DG alone proved to be ineffective as mono-therapy, suggesting a synergistic effect with CAR-T cells. Importantly, the same dose of 2DG able to enhance tumor cell recognition by CAR-T cells failed to increase elimination of healthy cells, supporting the safety of the strategy. Finally, when challenged in a pancreatic adenocarcinoma xenograft mouse model, the antitumor activity of CAR-T cells was significantly increased by 2DG and this effect was accompanied by a decrease in the expression of exhaustion and senescence markers. Notably, these results were confirmed when using CEA-CAR T cells and different tumour types, suggesting that the combined strategy can be successfully applied to multiple cancers with different CAR specificities.

In conclusion, the present results indicate that tumor glycosylation modulates CAR-T cell efficacy and support the combination of CAR-T cells with a glycosylation inhibitor such as the de-glycosylation agent 2DG, for a successful immunotherapy against solid or hematopoietic tumors.

In particular to investigate if sugar chains may be sterically hulking for CAR-T cell targeting, the inventors generated N-glycosylation-defective pancreatic tumor cell lines by knocking-out the expression of the glycosyltransferase Mgat5 using the CRISPR-Cas9 technology. As model antigens for CAR targeting, they focused on CD44v6 and CEA since they are both heavily glycosylated proteins over-expressed on a wide variety of solid tumors, including pancreatic adenocarcinoma. Strikingly, hampering N-glycosylation resulted in a dramatic increase of tumor targeting by both CD44v6 and CEA CAR-T cells. This effect associated with improved CAR-T cell activation, suggesting more proficient antigen engagement. These findings were further confirmed using the N-glycosylation inhibitor tunicamycin. To exploit this mechanism in order to increase the efficacy of CAR-T cells against solid tumors, the inventors sought to block tumor N-glycosylation with the glucose/mannose analogue 2-Deoxy-D-glucose (2DG). Similarly to glycosylation knocked-out cells, treatment with 2DG also sensitized tumor cells to recognition by CAR-T cells, significantly increasing their elimination. Notably, 2DG alone proved to be ineffective, further suggesting a synergistic effect with CAR-T cells. Mechanistically, both Mgat5 knock-out and 2DG decreased PHA-L lectin staining on pancreatic tumor cells, showing that treatment effectively reduces N-glycan branching. Similar results were also confirmed by Western blot looking at the presence of de-glycosylated proteins on tumor cell surface after 2DG treatment. Next, the inventors challenged the combined approach in a pancreatic adenocarcinoma xenograft mouse model. According to in vitro data, mice bearing an high tumor burden and receiving CAR-T cells highly benefited from 2DG administration (5-fold less tumor at 7d, p<0,05), which conversely was unable to mediate any antitumor effect alone. Interestingly, improved antitumor activity was accompanied by a decrease in the frequency of CAR-T cells co-expressing exhaustion and senescence markers such as TIM-3, LAG-3, PD-1 and CD57. Thanks to metabolic deregulation (Warburg effect), 2DG is expected to selectively accumulate in cancer cells compared to healthy tissues, supporting the safety of the combined approach. Accordingly, it was observed that the same doses of 2DG able to enhance tumor recognition by CAR-T cells failed to induce protein de-glycosylation and to increase the elimination of healthy cells, such as keratinocytes. Finally, cancer cell lines from several solid and hematopoietic tumors were found highly N-glycosylated, and their killing was significantly improved by combining CAR-T cells with the glycosylation inhibitor 2DG.

The present results indicate that i) the glycosylation status of tumor cells regulates the efficacy of CAR-T cells and ii) combining CAR-T cells with a glycosylation inhibitor such as the de-glycosylation agent 2DG, which preferentially accumulates in tumor masses, represents a successful immunotherapy against tumors, in particular solid tumors.

In the present invention it was surprisingly found that the glycosylation status of tumour may influence their susceptibility to killing by immunotherapy, preferably cell-based immunotherapy, more preferably CAR-T cell therapy and that de-glycosylating or glycosylation inhibitor agents, like 2-deoxy-D-glucose (2DG), can synergistically improve the antitumor efficacy of said immunotherapy, such as CAR-T cells.

Chimeric antigen receptors are synthetic biology molecules commonly constructed by fusing an antigen-binding moiety derived from a tumour-reactive monoclonal antibody with intracellular signalling domains derived from T lymphocytes. Solid malignancies are characterized by a wide range of glycosylation alterations, where glycans have the potential to modulate tumour interaction with the immune system. This invention is based on the finding that treating tumour with de-glycosylating agents (or glycosylation inhibitors) increase their recognition and killing by CAR-T cells, thus improving the final therapeutic outcome. 2-Deoxy-D-glucose (2DG) is a glucose analogue able to inhibit the first critical steps of glycolysis. Moreover, since it structurally mimics D-mannose, 2DG is also able to inhibit N-glycosylation. Thanks to metabolic deregulation in tumour cells (Warburg effect), 2DG is expected to selectively accumulate in cancer cells and has been proposed as a candidate for anticancer therapy. Recent clinical trials confirmed that, despite low antitumor activity, the administration of 2DG alone or combined with other anticancer therapies, such as chemotherapy and radiotherapy, was safe and well tolerated by patients (Zhang D, Cancer Letters 2014; Xi H, IUBMB Life 2014). The inventors show that 2DG significantly increased tumour cell recognition and elimination by CAR-T cells. Notably, this effect is not simply additive but synergistic, since it was above what could be expected from the individual activity of 2DG (which was minimal) and CAR-T cells alone. 2DG also displays low toxicity to normal cells due to preferential accumulation in tumour cells. However, de-glycosylating agents or a glycosylation inhibitor other than 2DG can also be implemented. Moreover, selective delivery of said agents or inhibitors to tumor or is achieved, for example by the use of nanoparticles functionalized for specific cell targeting.

Since CAR-T cells and monoclonal antibodies share the recognition moiety, glycosylation inhibition is expected to positively influence also antigen recognition by monoclonal antibody therapy. Moreover, it has been reported that the glycosylation status of checkpoint molecules expressed on tumor cells, like PDL1, is important for their immunosuppressive function via receptor binding on T cells (Li et al, Cancer Cell 33, 187-201, 2018). This suggests that inhibition of tumor or infected cell glycosylation with 2DG could generally unleash T-cell based therapies. Then, the present invention provides at least one glycosylation inhibitor and at least one CAR cell therapy for use in the treatment and/or prevention of cancer.

Preferably said at least one glycosylation inhibitor improves therapeutic potential of said CAR cell therapy and/or improves CAR cell activation and/or increases antigen engagement and/or sensitizes tumour cells to recognition by the CAR cell therapy and/or increases elimination of tumour cells.

The present invention provides at least one glycosylation inhibitor for use in increasing tumour cell killing wherein said tumour cell is exposed to at least one CAR cell therapy or wherein said tumor cell has been exposed to at least one CAR cell therapy.

Preferably the at least CAR cell therapy is a CAR-T cell therapy or a CAR-NK cell therapy, preferably the T cell is autologous or allogeneic T cell.

In a preferred embodiment said glycosylation inhibitor is selected from the group consisting of: a O-glycosylation inhibitor, a N-glycosylation inhibitor, a P-glycosylation inhibitor, a C-glycosylation inhibitor, a S-glycosylation inhibitor or a combination thereof.

Preferably said glycosylation inhibitor is selected from the group consisting of: a mannose analog, 2-deoxyglucose, 3-deoxy-3-fluoroglucosamine, 4-deoxy-4-fluoroglucosamine, 2-deoxy-2-fluoro-glucose, 2-deoxy-2-fluoro-mannose, 6-deoxy-6-fluoro-N-acetylglucosamine, 2-deoxy-2-fluorofucose, and 3-fluoro sialic acid tunicamycin, castanospermine, australine, deoxynojirimycin, swainsonine, deoxymannojirimycin, kifunensin, mannostatin, neuraminidase inhibitors, inhibitors of glycosyltransferases.

Preferably the at least one glycosylation inhibitor for use as above defined further comprises a therapeutic agent, preferably said therapeutic agent is an antibody, preferably a checkpoint inhibitor antibody. Preferred antibodies are defined below.

Checkpoint inhibitor therapy is a form of cancer immunotherapy. The therapy targets immune checkpoints, key regulators of the immune system that stimulate or inhibit its actions, which tumors can use to protect themselves from attacks by the immune system. Checkpoint therapy can block inhibitory checkpoints, restoring immune system function. The first anti-cancer drug targeting an immune checkpoint was ipilimumab, a CTLA4 blocker approved in the United States in 2011.

Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumor.

In a preferred embodiment the glycosylation inhibitor is 2-deoxyglucose.

Preferably the at least one glycosylation inhibitor for use as above defined is for the treatment of a solid or haematopoietic or lymphoid tumor. Preferably the solid tumor is selected from the group consisting of: colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angio genesis, spinal axis tumor, brain stem glioma, pituitary adenoma, Kaposi's sarcoma, epidermoid cancer, squamous cell cancer, environmentally induced cancers, combinations of said cancers, and metastatic lesions of said cancers.

Preferably the haematopoietic or lymphoid tumor is selected from the group consisting of: chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia, combinations of said cancers, and metastatic lesions of said cancers.

In a preferred embodiment the at least one glycosylation inhibitor is administered prior to the CAR cell therapy or the at least one glycosylation inhibitor is administered concomitantly to the CAR cell therapy.

The invention also provides a composition comprising at least one glycosylation inhibitor and a CAR cell therapy for medical use.

Preferably said at least one glycosylation inhibitor improves therapeutic potential of said CAR cell therapy and/or improves CAR cell activation and/or increases antigen engagement and/or sensitizes tumour cells to recognition by the CAR cell therapy and/or increases elimination of tumour cells.

The present invention provides a composition comprising at least one glycosylation inhibitor for use in increasing tumour cell killing wherein said tumour cell is exposed to at least one CAR cell therapy or wherein said tumor cell has been exposed to at least one CAR cell therapy.

Preferably the glycosylation inhibitor is 2DG and the CAR cell therapy is a CAR-T cell therapy.

The invention also provides a isolated population or subpopulation of CAR-T cells or an isolated CAR-T cell that is contacted with at least one glycosylation inhibitor.

Preferably said at least one glycosylation inhibitor improves therapeutic potential of said CAR cell therapy or increases the sensibility of the subject to said CAR cell therapy.

The invention also provides at least one glycosylation inhibitor for use in combination with a CAR cell therapy in a subject wherein said at least one glycosylation inhibitor increases tumor cell recognition by said CAR cell therapy and/or wherein said at least one glycosylation inhibitor improves elimination of tumor cells.

In another aspect, the invention provides a CAR therapy including a cell, e.g., a population of immune effector cells, including (e.g., expressing) a chimeric antigen receptor (CAR) for use in combination with an inhibitor of glycosylation in treating a subject having a disease associated with expression of a tumor antigen (e.g., a subject having a cancer (e.g., a solid or hemtopoietic or blood tumor or a tumor associated with tumor-associated macrophages)). The CAR includes a tumor antigen binding domain (e.g., the tumor antigen binding domain of the CAR binds to CD19 or CD123), a transmembrane domain, and an intracellular signaling domain.

In embodiments, including in any of the aforementioned aspects and embodiments, the cell is a T cell or an NK cell. In embodiments, the T cell is an autologous or allogeneic T cell.

In embodiments, the CAR therapy and the inhibitor of glycosylation are administered sequentially. In embodiments, including in any of the aforementioned aspects and embodiments, the inhibitor of glycosylation is administered prior to the CAR therapy. In embodiments, including in any of the aforementioned aspects and embodiments, the inhibitor of glycosylation and the CAR therapy are administered simultaneously or concurrently.

In embodiments, including in any of the aforementioned aspects and embodiments, the CAR therapy is administered as (a) single infusion or (b) multiple infusions (e.g., a single dose split into multiple infusions), and inhibitor of glycosylation is administered as (a) a single dose, or (b) multiple doses (e.g., a first and second, and optionally one or more subsequent doses).

In embodiments, including in any of the aforementioned aspects and embodiments, a dose of the CAR therapy is administered after (e.g., at least 1 day, 2 days, 3 days, 4 days, 5 days, 6 days, 7 days, 1 week, 2 weeks, 3 weeks, 4 weeks, 5 weeks, or more, after) administration of a first dose of the inhibitor of glycosylation, e.g., and before administration of the second dose of the inhibitor.

In embodiments, including in any of the aforementioned aspects and embodiments, a dose of the CAR therapy is administered concurrently with (e.g., within 2 days (e.g., within 2 days, 1 day, 24 hours, 12 hours, 6 hours, 4 hours, 2 hours, or less) the administration of a first dose of the inhibitor of glycosylation.

In embodiments, including in any of the aforementioned aspects and embodiments, one or more subsequent doses of the inhibitor of glycosylation are administered after a second dose of the inhibitor of glycosylation.

In embodiments, including in any of the aforementioned aspects and embodiments, the inhibitor of glycosylation is administered in more than one dose, and the doses are administered twice a day (BID), once a day, once a week, once every 14 days, or once every month.

In embodiments, including in any of the aforementioned aspects and embodiments, the administering of the inhibitor of glycosylation includes multiple doses including a duration of at least 7 days, e.g., at least 7 days, 8 days, 9 days, 10 days, 1 week, 2 weeks, 3 weeks, 4 weeks, 5 weeks, 6 weeks, 1 month, 2 months, 3 months, 4 months, 5 months, 6 months, 7 months, 8 months, or more.

In embodiments, including in any of the aforementioned aspects and embodiments, the CAR therapy is administered at a dose comprising at least about 5×10⁶, 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells, e.g., CAR positive cells.

In another aspect, the invention provides a method for stimulating a T cell-mediated immune response to a solid tumor cell in a mammal, the method including administering to a mammal an effective amount of a composition of the previous aspects.

In another aspect, the invention provides a method of providing an anti-tumor, e.g., an anti-solid or hematopoietic tumor, immunity in a mammal, including administering to the mammal an effective amount of the composition

In another aspect, the invention provides a method of treating a mammal having a disease associated with expression of a tumor antigen, e.g., a solid tumor antigen, said method including administering an effective amount of the composition of the previous aspects.

In embodiments, including in any of the method embodiments above, the cell, e.g., the population of immune effector cells, and the inhibitor of glycosylation are provided for separate administration (e.g., in two separate compositions). In other embodiments, including in any of the method embodiments above, the cell, e.g., the population of immune effector cells, and the inhibitor of glycosylation are provided for simultaneous administration (e.g., in one composition).

The following aspects of the inhibitor of glycosylation may be utilized with any of the aforementioned aspects and embodiments.

Chemical tools to inhibit glycosylation comprise various types of inhibitors, including natural products, substrate-based tight-binding inhibitors, glycoside primers, inhibitors found through screening chemical libraries, and rationally designed inhibitors based on three-dimensional structures of enzymes.

Then a glycosylation inhibitor of the present invention comprises different classes of inhibitors (see table below): metabolic inhibitors, inhibitors of dolichol-PP-GlcNAc assembly, plant alkaloids as natural inhibitors of glycosidases, inhibitors of O-GalNac initiation of mucin-type glycans, inhibitors of O-GlcNAc modification, substarte analogs, glycoside primers, inhibitors of glycolipids and GPI anchors, Neuraminidase (Sialidase) Inhibitors, Sulfotransferase Inhibitors. These inhibitors are described in “Chemical Tools for Inhibiting Glycosylation—Essentials of Glycobiology-NCBI Bookshelf”, Varki A, Cummings R D, Esko J D, et al., editors. Essentials of Glycobiology [Internet]. 3rd edition. Cold Spring Harbor (N.Y.): Cold Spring Harbor Laboratory Press; 2015-2017. doi: 10.1101/glycobiology.3e.055, in particular Chapter 55 Chemical Tools for Inhibiting Glycosylation, Jeffrey D. Esko, Carolyn Bertozzi, and Ronald L. Schnaar, incorporated in its entirety by reference.

| Class of | |
|---|---|
| inhibitor/modulator | Target |
| Metabolic inhibitors | steps involved in formation of common intermediates such as PAPS or nucleotide sugars |
| Tunicamycin | N-linked glycosylation through inhibition of dolichol-PP-GlcNAc formation; peptidoglycan |
| | biosynthesis through inhibition of undecaprenyl-PP-GlcNAc assembly |
| Plant alkaloids | N-linked glycosylation through inhibition of processing glycosidases |
| Substrate analogs | specific glycosyltansferases or glycosidases |
| Glycoside primers | glycosylation pathways by diverting the assembly of glycans from endogenous acceptors to |
| | exogenous primers |

A number of inhibitors have been described that block glycosylation by interfering with the metabolism of common precursors or intracellular transport activities. Some of these compounds act indirectly by impeding the transit of proteins between the endoplasmic reticulum (ER), Golgi, and trans-Golgi network. For example, the fungal metabolite brefeldin A causes retrograde transport of Golgi components located proximal to the trans-Golgi network back to the ER. Thus, treating cells with brefeldin A separates enzymes located in the trans-Golgi network from those found in the ER and Golgi, and uncouples the assembly of the core structures of some glycans from later reactions, such as sialylation or sulfation. The drug can be used to examine if two pathways reside in the same compartment or share the same enzymes. Because the localization and array of the enzymes vary considerably in different cell types, extrapolating the effects of brefeldin A from one system to another is often difficult.

Some inhibitors act at key steps in intermediary metabolism in which precursors involved in glycosylation are formed. For example, a glutamine analog, 6-diazo-5-oxo-L-norleucine (DON), blocks glutamine: fructose-6-phosphate amidotransferase, the enzyme of the hexosamine biosynthetic pathway that forms glucosamine from fructose and glutamine. Depressing glucosamine production in this way has a pleiotropic effect on glycan assembly because all of the major families contain N-acetylglucosamine or N-acetylgalactosamine. DON also affects other glutamine using enzymes and, therefore, care should be taken to limit nonspecific side effects. Chlorate is another type of general inhibitor that blocks sulfation. The chlorate anion (ClO) is an analog of sulfate (SO) and it forms an abortive complex with the sulfurylase involved in the formation of phosphoadenosine-5′-phosphosulfate (PAPS), the active sulfate donor for all known sulfation reactions. Thus, treating cells with chlorate (usually 10-30 mM) inhibits sulfation by >90%, but the effect is not specific for any particular class of glycan or sulfation reaction (e.g., tyrosine sulfation is also affected).

A number of sugar analogs have been made, showing selective inhibition of glycosylation:

2-Deoxyglucose and fluorinated analogs of sugars (3-deoxy-3-fluoroglucosamine, 4-deoxy-4-fluoroglucosamine, 6-deoxy-6-fluoro-N-acetylglucosamine, 2-deoxy-2-fluoroglucose, 2-deoxy-2-fluoromannose, 2-deoxy-2-fluorofucose, and 3-fluorosialic acid) inhibit glycoprotein biosynthesis. Early studies of 2-deoxyglucose showed that the analog was converted to UDP-2-deoxyglucose as well as to GDP-2-deoxyglucose and dolichol-P-2-deoxyglucose. Inhibition of glycoprotein formation apparently occurs as a result of accumulation of various dolichol oligosaccharides containing 2-deoxyglucose, which cannot be elongated or transferred to glycoproteins normally. Similarly, 4-deoxy-N-acetylglucosamine is converted to UDP-4-deoxy-N-acetylglucosamine, resulting in inhibition of heparan sulfate formation without incorporation of the analog. 4-Deoxy-xylose also inhibits glycosaminoglycan assembly presumably by competing with naturally occurring xylosylated substrates.

Further glycosylation inhibitors of the present invention include:

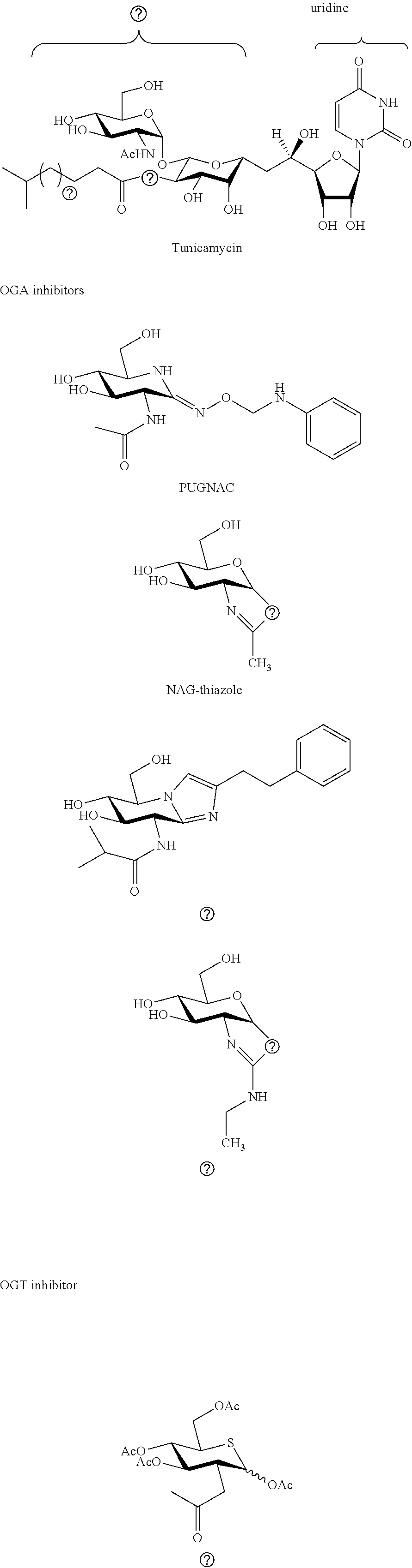

Inhibitors of O-GlcNAc-specific β-hexosaminidase (OGA) and O-GlcNAc transferase (OGT).

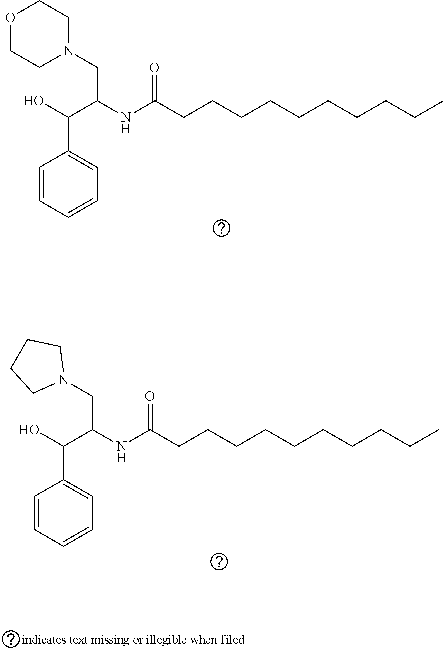 [SMILES: CCCCCCCCCCC(=O)NC(CN1CCOCC1)C(O)c1ccccc1 ; CCCCCCCCCCC(=O)NC(CN1CCCC1)C(O)c1ccccc1]

Inhibitors of glycosphingolipid formation. These analogs are potent inhibitors of the glucosyltransferase that initiates glycosphingolipid formation.

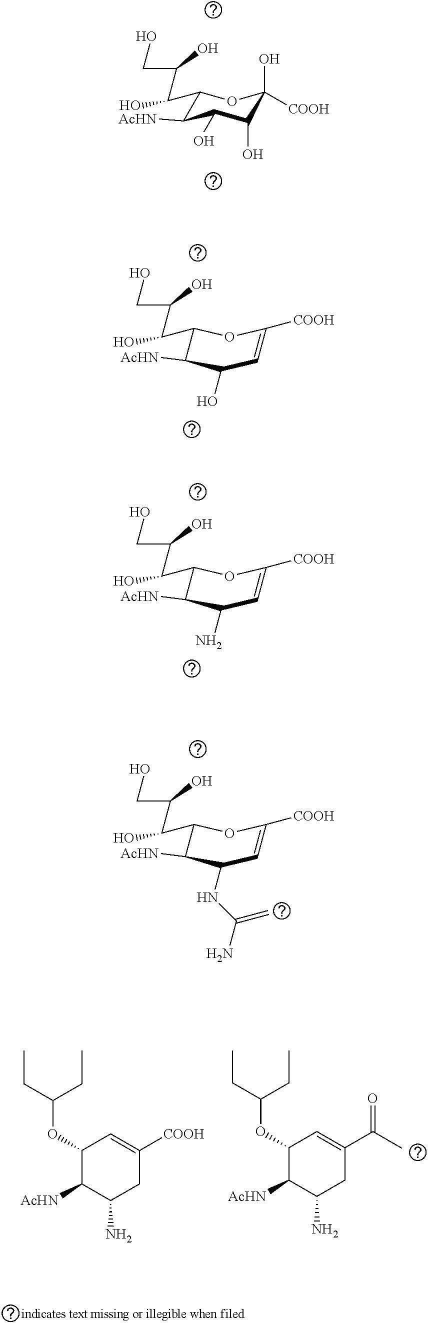

Structure of influenza neuraminidase inhibitors. Chemical structure of NeuSAc, NANA; 2-deoxy-2,3-dehydro-Nacetyl neuraminic acid, DANA; 4-amino-DANA; 4-guanidino-DANA (Relenza, zanamivir); (3R, 4R, 5S)-4-acetamido-5-amino-3-(1-ethylpropoxyl)-1-cyclohexane-1-carboxylic acid ethyl ester (GS4104, Tamiflu, oseltamivir). DANA is thought to resemble the transition state in hydrolysis, and addition of the guanidinium group in Relenza provides higher affinity binding to the active site. The ethyl ester in Tamiflu enhances oral availability and then is quickly removed in the body by nonspecific esterases.

Examples of alkaloids that inhibit glycosidases involved in N-linked glycan biosynthesis:

| Alkaloid | Source | Target |
|---|---|---|
| [symbol] | 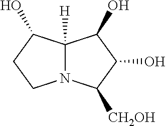 [SMILES: OC[C@@H]1[C@@H](O)[C@H](O)[C@@H]2[C@@H](O)CCN21] | α-gluco [symbol] |
| Cas [symbol] | 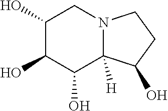 [SMILES: O[C@@H]1[C@@H](O)[C@H](O)CN2CC[C@@H](O)[C@@H]12] | α-glucosidase I and II |
| Deoxy [symbol] | 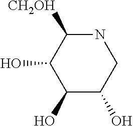 [SMILES: OC[C@H]1NC[C@H](O)[C@@H](O)[C@@H]1O] | α-gluco [symbol] |
| Deoxy [symbol] | 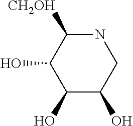 [SMILES: OC[C@H]1NC[C@@H](O)[C@@H](O)[C@@H]1O] | α- [symbol] |
| Kif [symbol] | 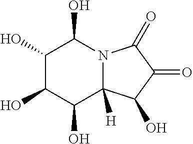 [SMILES: O=C1C(=O)N2[C@@H]([C@@H](O)[C@@H](O)[C@H](O)[C@H]2O)[C@@H]1O] | α- [symbol] |
| [symbol] | 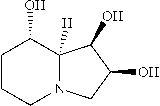 [SMILES: O[C@@H]1[C@@H]2[C@@H](O)CCCN2C[C@@H]1O] | α- [symbol] |
| [symbol] | 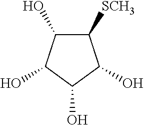 [SMILES: CS[C@H]1[C@H](O)[C@H](O)[C@H](O)[C@@H]1O] | α- [symbol] |

[symbol]

indicates data missing or illegible when filed

Synthetic substrate-based inhibitors of glycosyltransferases

| Enzyme | Substrate | Inhibitor |
|---|---|---|
| α2FucT | β3GlcNAcβ-O—R | 2-deoxyGalβ3GlcNAcβ-O—R |
| β4GalT | GlcNAcβ3Galβ-O—R | 6-thioGlcNAcβ3Galβ-O—Me |
| α3GalT | Galβ4GlcNAcβ-O—R | 3-aminoGalβ4GlcNAcβ-O—R |
| β6GlcNAcT | Galβ3GalNAcα-O—R | Galβ3(6-deoxy)GalNAcα-O—R |
| β6GlcNAcT-V | GlcNAcβ2Manα6Glcβ-O—R | GlcNAcβ2(6-deoxy)Manα6Glc β-O—R |
| β6GlcNAcT-V | GlcNAcβ2Manα6Glcβ-O—R | GlcNAcβ2(4-O-methyl)Manα6 Glcβ-O—R |
| β6GlcNAcT-V | GlcNAcβ2Manα6Glcβ-O—R | GlcNAcβ2(6-deoxy,4-O—Me)Manα6Glcβ-O—R |
| α6SialylT | Galβ4GlcNAcβ-O—R | 6-deoxyGalβ2GlcNAcβ-O—R |
| α3GalNAcT-A | Fucα2Galβ-O—R | Fucα2(3-deoxy)Galβ-O—R |
| α3GalNAcT-A | Fucα2Galβ-O—R | Fucα2(3-amino)Galβ-O—R |

In the present invention cancer immunotherapy (sometimes called immuno-oncology, abbreviated IO) is the use of the immune system to treat cancer Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines.

Among these, multiple antibody therapies are approved in various jurisdictions to treat a wide range of cancers. Antibodies are proteins produced by the immune system that bind to a target antigen on the cell surface. The immune system normally uses them to fight pathogens. Each antibody is specific to one or a few proteins. Those that bind to tumor antigens treat cancer. Cell surface receptors are common targets for antibody therapies and include CD20, CD274 and CD279. Once bound to a cancer antigen, antibodies can induce antibody-dependent cell-mediated cytotoxicity, activate the complement system, or prevent a receptor from interacting with its ligand, all of which can lead to cell death. Approved antibodies include alemtuzumab, ipilimumab, nivolumab, ofatumumab and rituximab

Active cellular therapies usually involve the removal of immune cells from the blood or from a tumor. Those specific for the tumor are cultured and returned to the patient where they attack the tumor; alternatively, immune cells can be genetically engineered to express a tumor-specific receptor, cultured and returned to the patient. Cell types that can be used in this way are natural killer cells, lymphokine-activated killer cells, cytotoxic T cells and dendritic cells.

Interleukin-2 and interferon-α are cytokines, proteins that regulate and coordinate the behaviour of the immune system. They have the ability to enhance anti-tumor activity and thus can be used as passive cancer treatments. Interferon-α is used in the treatment of hairy-cell leukaemia, AIDS-related Kaposi's sarcoma, follicular lymphoma, chronic myeloid leukaemia and malignant melanoma. Interleukin-2 is used in the treatment of malignant melanoma and renal cell carcinoma.

Adoptive T-Cell Therapy

Cancer specific T-cells can be obtained by fragmentation and isolation of tumour infiltrating lymphocytes, or by genetically engineering cells from peripheral blood. The cells are activated and grown prior to transfusion into the recipient (tumour bearer).

Adoptive T-cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

As of 2014, multiple ACT clinical trials were underway. Importantly, one study from 2018 showed that clinical responses can be obtained in patients with metastatic melanoma resistant to multiple previous immunotherapies.

The first 2 adoptive T-cell therapies, tisagenlecleucel and axicabtagene ciloleucel, were approved by the FDA in 2017.

Another approach is adoptive transfer of haploidentical γδ T cells or NK cells from a healthy donor. The major advantage of this approach is that these cells do not cause GVHD. The disadvantage is frequently impaired function of the transferred cells.

CAR-T Cell Therapy

Chimeric Antigen Receptors

“Chimeric antigen receptor” or “CAR” or “CARs” refers to engineered receptors which can confer an antigen specificity onto cells (for example T cells). CARs are also known as artificial T-cell receptors, chimeric T-cell receptors or chimeric immunoreceptors. Preferably the CARs of the invention comprise an antigen-specific targeting region, an extracellular domain, a transmembrane domain, optionally one or more co-stimulatory domains, and an intracellular signaling domain.

Antigen-Specific Targeting Domain

The antigen-specific targeting domain provides the CAR with the ability to bind to the target antigen of interest. The antigen-specific targeting domain preferably targets an antigen of clinical interest against which it would be desirable to trigger an effector immune response that results in tumor killing.

The antigen-specific targeting domain may be any protein or peptide that possesses the ability to specifically recognize and bind to a biological molecule (e.g., a cell surface receptor or tumor protein, or a component thereof). The antigen-specific targeting domain includes any naturally occurring, synthetic, semi-synthetic, or recombinantly produced binding partner for a biological molecule of interest.

Illustrative antigen-specific targeting domains include antibodies or antibody fragments or derivatives, extracellular domains of receptors, ligands for cell surface molecules/receptors, or receptor binding domains thereof, and tumor binding proteins.

In a preferred embodiment, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a camelid antibody (VHH).

In a preferred embodiment, the binding domain is a single chain antibody (scFv). The scFv may be murine, human or humanized scFv.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs.

“Heavy chain variable region” or “VH” refers to the fragment of the heavy chain of an antibody that contains three CDRs interposed between flanking stretches known as framework regions, which are more highly conserved than the CDRs and form a scaffold to support the CDRs.

“Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence.

Antibodies that specifically bind a tumor cell surface molecule can be prepared using methods well known in the art. Such methods include phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

Examples of antigens which may be targeted by the CAR of the invention include but are not limited to antigens expressed on cancer cells and antigens expressed on cells associated with various hematologic diseases, autoimmune diseases, inflammatory diseases and infectious diseases.

With respect to targeting domains that target cancer antigens, the selection of the targeting domain will depend on the type of cancer to be treated, and may target tumor antigens. A tumor sample from a subject may be characterized for the presence of certain biomarkers or cell surface markers. For example, breast cancer cells from a subject may be positive or negative for each of Her2Neu, Estrogen receptor, and/or the Progesterone receptor. A tumor antigen or cell surface molecule is selected that is found on the individual subject's tumor cells. Preferably the antigen-specific targeting domain targets a cell surface molecule that is found on tumor cells and is not substantially found on normal tissues, or restricted in its expression to non-vital normal tissues.

Further antigens specific for cancer which may be targeted by a CAR include but are not limited to any one or more of mesothelin, EGFRvIII, TSHR, CD19, CD123, CD22, CD30, CD171, CS-1, CLL-1, CD33, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), MUC1, epidermal growth factor receptor (EGFR), NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRCSD, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1,

Antigens specific for inflammatory diseases which may be targeted by the CAR of the invention include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin α4, integrin α4β7, Lama glama, LFA-1 (CD11a), MEDI-528, myostatin, OX-40, rhuMAb β7, scleroscin, SOST, TGF β1, TNF-a or VEGF-A.

Antigens specific for neuronal disorders which may be targeted by the CAR of the invention include but are not limited to any one or more of beta amyloid or MABT5102A.

Antigens specific for diabetes which may be targeted by the CAR of the invention include but are not limited to any one or more of L-1β or CD3. Other antigens specific for diabetes or other metabolic disorders will be apparent to those of skill in the art.

Antigens specific for cardiovascular diseases which may be targeted by the CARs of the invention include but are not limited to any one or more of C5, cardiac myosin, CD41 (integrin alpha-IIb), fibrin II, beta chain, ITGB2 (CD18) and sphingosine-1-phosphate.

Preferably, the antigen-specific binding domain specifically binds to a tumor antigen. In a specific embodiment, the polynucleotide codes for a single chain Fv that specifically binds CD44v6 or CEA.

Co-Stimulatory Domain

The CAR also comprises one or more co-stimulatory domains. This domain may enhance cell proliferation, cell survival and development of memory cells.

Each co-stimulatory domain comprises the co-stimulatory domain of any one or more of, for example, a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CD5, ICAM-1, LFA-1 (CD11a/CD18), 4-1BB (CD137), B7-H3, CD5, ICAM-1, ICOS (CD278), GITR, BAFFR, LIGHT, HVEM (LIGHTR), KIRDS2, SLAMF7, NKp80 (KLRF1), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB 1, CD29, ITGB2, CD 18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD 19a, and a ligand that specifically binds with CD83. Additional co-stimulatory domains will be apparent to those of skill in the art.

Intracellular Signaling Domain

The CAR also comprises an intracellular signaling domain. This domain may be cytoplasmic and may transduce the effector function signal and direct the cell to perform its specialized function. Examples of intracellular signaling domains include, but are not limited to, ζ chain of the T-cell receptor or any of its homologs (e.g., η chain, FcεR1γ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may be human CD3 zeta chain, FcyRIII, FcsRI, cytoplasmic tails of Fc receptors, immunoreceptor tyrosine-based activation motif (ITAM) bearing cytoplasmic receptors or combinations thereof.

Preferable, the intracellular signaling domain comprises the intracellular signaling domain of human CD3 zeta chain.

Transmembrane Domain

The CAR also comprises a transmembrane domain. The transmembrane domain may comprise the transmembrane sequence from any protein which has a transmembrane domain, including any of the type I, type II or type III transmembrane proteins. The transmembrane domain of the CAR of the invention may also comprise an artificial hydrophobic sequence. The transmembrane domains of the CARs of the invention may be selected so as not to dimerize. Additional transmembrane domains will be apparent to those of skill in the art. Examples of transmembrane (TM) regions used in CAR constructs are: 1) The CD28 TM region (Pule et al, Mol Ther, 2005, November; 12(5):933-41; Brentjens et al, CCR, 2007, September 15; 13(18 Pt 1):5426-35; Casucci et al, Blood, 2013, Nov. 14; 122(20):3461-72.); 2) The OX40 TM region (Pule et al, Mol Ther, 2005, November; 12(5):933-41); 3) The 41BB TM region (Brentjens et al, CCR, 2007, Sep. 15; 13(18 Pt 1):5426-35); 4) The CD3 zeta TM region (Pule et al, Mol Ther, 2005, November; 12(5):933-41; Savoldo B, Blood, 2009, Jun. 18; 113(25):6392-402.); 5) The CD8a TM region (Maher et al, Nat Biotechnol, 2002, January; 20(1):70-5.; Imai C, Leukemia, 2004, April; 18(4):676-84; Brentjens et al, CCR, 2007, Sep. 15; 13(18 Pt 1):5426-35; Milone et al, Mol Ther, 2009, August; 17(8):1453-64.).

Approved Drugs

Tisagenlecleucel (Kymriah), a chimeric antigen receptor (CAR-T) therapy, was approved by FDA in 2017 to treat acute lymphoblastic leukemia. This treatment removes CD19 positive cells (B-cells) from the body (including the diseased cells, but also normal antibody producing cells). Axicabtagene ciloleucel (Yescarta) is another CAR-T therapeutic, approved in 2017 for treatment of diffuse large B-cell lymphoma.

Antibody Therapy

Antibodies are a key component of the adaptive immune response, playing a central role in both recognizing foreign antigens and stimulating an immune response. Antibodies are Y-shaped proteins produced by some B cells and are composed of two regions: an antigen-binding fragment (Fab), which binds to antigens, and a Fragment crystallizable (Fc) region, which interacts with so-called Fc receptors that are expressed on the surface of different immune cell types including macrophages, neutrophils and NK cells. Many immunotherapeutic regimens involve antibodies. Monoclonal antibody technology engineers and generates antibodies against specific antigens, such as those present on tumor surfaces.

Antibody Types

Conjugation

Two types are used in cancer treatments:

Naked monoclonal antibodies are antibodies without added elements. Most antibody therapies use this antibody type.

Conjugated monoclonal antibodies are joined to another molecule, which is either cytotoxic or radioactive. The toxic chemicals are those typically used as chemotherapy drugs, but other toxins can be used. The antibody binds to specific antigens on cancer cell surfaces, directing the therapy to the tumor. Radioactive compound-linked antibodies are referred to as radiolabelled. Chemolabelled or immunotoxins antibodies are tagged with chemotherapeutic molecules or toxins, respectively.

Fc Regions

Fc's ability to bind Fc receptors is important because it allows antibodies to activate the immune system. Fc regions are varied: they exist in numerous subtypes and can be further modified, for example with the addition of sugars in a process called glycosylation. Changes in the Fc region can alter an antibody's ability to engage Fc receptors and, by extension, will determine the type of immune response that the antibody triggers. Many cancer immunotherapy drugs, including PD-1 and PD-L1 inhibitors, are antibodies. For example, immune checkpoint blockers targeting PD-1 are antibodies designed to bind PD-1 expressed by T cells and reactivate these cells to eliminate tumors. Anti-PD-1 drugs contain not only an Fab region that binds PD-1 but also an Fc region. Experimental work indicates that the Fc portion of cancer immunotherapy drugs can affect the outcome of treatment. For example, anti-PD-1 drugs with Fc regions that bind inhibitory Fc receptors can have decreased therapeutic efficacy Imaging studies have further shown that the Fc region of anti-PD-1 drugs can bind Fc receptors expressed by tumor-associated macrophages. This process removes the drugs from their intended targets (i.e. PD-1 molecules expressed on the surface of T cells) and limits therapeutic efficacy. Furthermore, antibodies targeting the co-stimulatory protein CD40 require engagement with selective Fc receptors for optimal therapeutic efficacy. Together, these studies underscore the importance of Fc status in antibody-based immune checkpoint targeting strategies.

Human/Non-Human Balance

Antibodies are also referred to as murine, chimeric, humanized and human. Murine antibodies are from a different species and carry a risk of immune reaction. Chimeric antibodies attempt to reduce murine antibodies' immunogenicity by replacing part of the antibody with the corresponding human counterpart, known as the constant region. Humanized antibodies are almost completely human; only the complementarity determining regions of the variable regions are derived from murine sources. Human antibodies have been produced using unmodified human DNA.

Antibody-dependent cell-mediated cytotoxicity. When the Fc receptors on natural killer (NK) cells interact with Fc regions of antibodies bound to cancer cells, the NK cell releases perforin and granzyme, leading to cancer cell apoptosis.

Cell Death Mechanisms

Antibody-Dependent Cell-Mediated Cytotoxicity (ADCC)

Antibody-dependent cell-mediated cytotoxicity (ADCC) requires antibodies to bind to target cell surfaces. Antibodies are formed of a binding region (Fab) and the Fc region that can be detected by immune system cells via their Fc surface receptors. Fc receptors are found on many immune system cells, including natural killer cells. When natural killer cells encounter antibody-coated cells, the latter's Fc regions interact with their Fc receptors, releasing perforin and granzyme B to kill the tumor cell. Examples include Rituximab, Ofatumumab and Alemtuzumab. Antibodies under development have altered Fc regions that have higher affinity for a specific type of Fc receptor, FcγRIIIA, which can dramatically increase effectiveness.

Complement

The complement system includes blood proteins that can cause cell death after an antibody binds to the cell surface (the classical complement pathway, among the ways of complement activation). Generally the system deals with foreign pathogens, but can be activated with therapeutic antibodies in cancer. The system can be triggered if the antibody is chimeric, humanized or human; as long as it contains the IgG1 Fc region. Complement can lead to cell death by activation of the membrane attack complex, known as complement-dependent cytotoxicity; enhancement of antibody-dependent cell-mediated cytotoxicity; and CR3-dependent cellular cytotoxicity. Complement-dependent cytotoxicity occurs when antibodies bind to the cancer cell surface, the Cl complex binds to these antibodies and subsequently protein pores are formed in the cancer cell membrane.

FDA-Approved Antibodies

Cancer Immunotherapy: Monoclonal Antibodies

| | | | | Approval | |
|---|---|---|---|---|---|
| Antibody | Brand name | Type | Target | date | Approved treatment(s) |
| Alemtuzumab | Campath | humanized | CD52 | 2001 | B-cell chronic lymphocytic leukemia |
| | | | | | (CLL) |
| Atezolizumab | Tecentriq | humanized | PD-L1 | 2016 | bladder cancer |
| Avelumab | Bavencio | human | PD-L1 | 2017 | metastatic Merkel cell carcinoma |
| Ipilimumab | Yervoy | human | CTLA4 | 2011 | metastatic melanoma |
| Ofatumumab | Arzerra | human | CD20 | 2009 | refractory CLL |
| Nivolumab | Opdivo | human | PD-1 | 2014 | unresectable or metastatic |
| | | | | | melanoma, squamous non-small cell |
| | | | | | lung cancer, Renal cell carcinoma, |
| | | | | | colorectal cancer, hepatocellular |
| | | | | | carcinoma, classical Hodgkin |
| | | | | | lymphoma |
| Pembrolizumab | Keytruda | humanized | PD-1 | 2014 | metastatic melanoma |
| Rituximab | Rituxan, | chimeric | CD20 | 1997 | non-Hodgkin lymphoma |
| | Mabthera | | | | |
| Durvalumab | Imfinzi | human | PD-L1 | 2017 | bladder cancer, non-small cell lung |
| | | | | | cancer |

Alemtuzumab (Campeth-1H) is an anti-CD52 humanized IgG1 monoclonal antibody indicated for the treatment of fludarabine-refractory chronic lymphocytic leukemia (CLL), cutaneous T-cell lymphoma, peripheral T-cell lymphoma and T-cell prolymphocytic leukemia. CD52 is found on >95% of peripheral blood lymphocytes (both T-cells and B-cells) and monocytes, but its function in lymphocytes is unknown. It binds to CD52 and initiates its cytotoxic effect by complement fixation and ADCC mechanisms. Due to the antibody target (cells of the immune system) common complications of alemtuzumab therapy are infection, toxicity and myelosuppression.

Atezolizumab

Durvalumab

Durvalumab (Imfinzi) is a human immunoglobulin G1 kappa (IgG1κ) monoclonal antibody that blocks the interaction of programmed cell death ligand 1 (PD-L1) with the PD-1 and CD80 (B7.1) molecules. Durvalumab is approved for the treatment of patients with locally advanced or metastatic urothelial carcinoma who have disease progression during or following platinum-containing chemotherapy and/or have disease progression within 12 months of neoadjuvant or adjuvant treatment with platinum-containing chemotherapy.

Ipilimumab

Ipilimumab (Yervoy) is a human IgG1 antibody that binds the surface protein CTLA4. In normal physiology T-cells are activated by two signals: the T-cell receptor binding to an antigen-MHC complex and T-cell surface receptor CD28 binding to CD80 or CD86 proteins. CTLA4 binds to CD80 or CD86, preventing the binding of CD28 to these surface proteins and therefore negatively regulates the activation of T-cells.

Active cytotoxic T-cells are required for the immune system to attack melanoma cells. Normally inhibited active melanoma-specific cytotoxic T-cells can produce an effective anti-tumor response. Ipilumumab can cause a shift in the ratio of regulatory T-cells to cytotoxic T-cells to increase the anti-tumor response. Regulatory T-cells inhibit other T-cells, which may benefit the tumor.

Nivolumab

Ofatumumab

Ofatumumab is a second generation human IgG1 antibody that binds to CD20. It is used in the treatment of chronic lymphocytic leukemia (CLL) because the cancerous cells of CLL are usually CD20-expressing B-cells. Unlike rituximab, which binds to a large loop of the CD20 protein, ofatumumab binds to a separate, small loop. This may explain their different characteristics. Compared to rituximab, ofatumumab induces complement-dependent cytotoxicity at a lower dose with less immunogenicity.

Pembrolizumab

Pembrolizumab is approved for the first-line treatment of patients with metastatic non-small cell lung cancer whose tumors have high PD-L1 expression as determined by an FDA-approved test.

Rituximab

Rituximab is a chimeric monoclonal IgG1 antibody specific for CD20, developed from its parent antibody Ibritumomab. As with ibritumomab, rituximab targets CD20, making it effective in treating certain B-cell malignancies. These include aggressive and indolent lymphomas such as diffuse large B-cell lymphoma and follicular lymphoma and leukaemias such as B-cell chronic lymphocytic leukaemia. Although the function of CD20 is relatively unknown, CD20 may be a calcium channel involved in B-cell activation. The antibody's mode of action is primarily through the induction of ADCC and complement-mediated cytotoxicity. Other mechanisms include apoptosis and cellular growth arrest. Rituximab also increases the sensitivity of cancerous B-cells to chemotherapy.

Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferon

Interferons are produced by the immune system. They are usually involved in anti-viral response, but also have use for cancer. They fall in three groups: type I (IFNα and IFNβ), type II (IFNγ) and type III (IFNκ). IFNα has been approved for use in hairy-cell leukaemia, AIDS-related Kaposi's sarcoma, follicular lymphoma, chronic myeloid leukaemia and melanoma. Type I and II IFNs have been researched extensively and although both types promote anti-tumor immune system effects, only type I IFNs have been shown to be clinically effective. IFNκ shows promise for its anti-tumor effects in animal models.

Unlike type I IFNs, Interferon gamma is not approved yet for the treatment of any cancer. However, improved survival was observed when Interferon gamma was administrated to patients with bladder carcinoma and melanoma cancers. The most promising result was achieved in patients with stage 2 and 3 of ovarian carcinoma. The in vitro study of IFN-gamma in cancer cells is more extensive and results indicate anti-proliferative activity of IFN-gamma leading to the growth inhibition or cell death, generally induced by apoptosis but sometimes by autophagy.

Interleukin

Interleukins have an array of immune system effects. Interleukin-2 is used in the treatment of malignant melanoma and renal cell carcinoma. In normal physiology it promotes both effector T cells and T-regulatory cells, but its exact mechanism of action is unknown.

Combination Immunotherapy

Combining various immunotherapies such as PD1 and CTLA4 inhibitors can enhance anti-tumor response leading to durable responses.

Combining ablation therapy of tumors with immunotherapy enhances the immunostimulating response and has synergistic effects for curative metastatic cancer treatment.

Combining checkpoint immunotherapies with pharmaceutical agents has the potential to improve response, and such combination therapies are a highly investigated area of clinical investigation Immunostimulatory drugs such as CSF-1R inhibitors and TLR agonists have been particularly effective in this setting.

Polysaccharide-K

Japan's Ministry of Health, Labour and Welfare approved the use of polysaccharide-K extracted from the mushroom, Coriolus versicolor, in the 1980s, to stimulate the immune systems of patients undergoing chemotherapy. It is a dietary supplement in the US and other jurisdictions.

Anti-CD47 Therapy

Many tumor cells overexpress CD47 to escape immunosurveilance of host immune system. CD47 binds to its receptor signal regulatory protein alpha (SIRPα) and downregulate phagocytosis of tumor cell. Therefore, anti-CD47 therapy aims to restore clearance of tumor cells. Additionally, growing evidence supports the employment of tumor antigen-specific T cell response in response to anti-CD47 therapy. A number of therapeutics is being developed, including anti-CD47 antibodies, engineered decoy receptors, anti-SIRPa antibodies and bispecific agents. As of 2017, wide range of solid and hematologic malignancies were being clinically tested.

Anti-GD2 Antibodies

The GD2 Ganglioside

Carbohydrate antigens on the surface of cells can be used as targets for immunotherapy. GD2 is a ganglioside found on the surface of many types of cancer cell including neuroblastoma, retinoblastoma, melanoma, small cell lung cancer, brain tumors, osteosarcoma, rhabdomyosarcoma, Ewing's sarcoma, liposarcoma, fibrosarcoma, leiomyosarcoma and other soft tissue sarcomas. It is not usually expressed on the surface of normal tissues, making it a good target for immunotherapy. As of 2014, clinical trials were underway.

Immune Checkpoints

Immune checkpoints affect immune system function Immune checkpoints can be stimulatory or inhibitory. Tumors can use these checkpoints to protect themselves from immune system attacks. Currently approved checkpoint therapies block inhibitory checkpoint receptors. Blockade of negative feedback signaling to immune cells thus results in an enhanced immune response against tumors.

One ligand-receptor interaction under investigation is the interaction between the transmembrane programmed cell death 1 protein (PDCD1, PD-1; also known as CD279) and its ligand, PD-1 ligand 1 (PD-L1, CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. PD-L1 on cancer cells also inhibits FAS- and interferon-dependent apoptosis, protecting cells from cytotoxic molecules produced by T cells. Antibodies that bind to either PD-1 or PD-L1 and therefore block the interaction may allow the T-cells to attack the tumor.

CTLA-4 Blockade

The first checkpoint antibody approved by the FDA was ipilimumab, approved in 2011 for treatment of melanoma. It blocks the immune checkpoint molecule CTLA-4. Clinical trials have also shown some benefits of anti-CTLA-4 therapy on lung cancer or pancreatic cancer, specifically in combination with other drugs. In on-going trials the combination of CTLA-4 blockade with PD-1 or PD-L1 inhibitors is tested on different types of cancer.

However, patients treated with check-point blockade (specifically CTLA-4 blocking antibodies), or a combination of check-point blocking antibodies, are at high risk of suffering from immune-related adverse events such as dermatologic, gastrointestinal, endocrine, or hepatic autoimmune reactions. These are most likely due to the breadth of the induced T-cell activation when anti-CTLA-4 antibodies are administered by injection in the blood stream.

Using a mouse model of bladder cancer, researchers have found that a local injection of a low dose anti-CTLA-4 in the tumour area had the same tumour inhibiting capacity as when the antibody was delivered in the blood. At the same time the levels of circulating antibodies were lower, suggesting that local administration of the anti-CTLA-4 therapy might result in fewer adverse events.

PD-1 Inhibitors

Initial clinical trial results with IgG4 PD1 antibody Nivolumab were published in 2010. It was approved in 2014. Nivolumab is approved to treat melanoma, lung cancer, kidney cancer, bladder cancer, head and neck cancer, and Hodgkin's lymphoma. A 2016 clinical trial for non-small cell lung cancer failed to meet its primary endpoint for treatment in the first line setting, but is FDA approved in subsequent lines of therapy. Pembrolizumab is another PD1 inhibitor that was approved by the FDA in 2014. Keytruda (Pembrolizumab) is approved to treat melanoma and lung cancer.

Antibody BGB-A317 is a PD-1 inhibitor (designed to not bind Fc gamma receptor I) in early clinical trials.

PD-L1 Inhibitors

In May 2016, PD-L1 inhibitor atezolizumab was approved for treating bladder cancer.

Anti-PD-L1 antibodies currently in development include avelumab and durvalumab, in addition to an affimer biotherapeutic.

Other

Other modes of enhancing immuno-therapy include targeting so-called intrinsic checkpoint blockades e.g. CISH.

Oncolytic Virus

An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy.

The potential of viruses as anti-cancer agents was first realized in the early twentieth century, although coordinated research efforts did not begin until the 1960s. A number of viruses including adenovirus, reovirus, measles, herpes simplex, Newcastle disease virus and vaccinia have now been clinically tested as oncolytic agents. T-Vec is the first FDA-approved oncolytic virus for the treatment of melanoma. A number of other oncolytic viruses are in Phase II-III development.

Polysaccharides

Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

Neoantigens

Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors. In non-small cell lung cancer patients treated with lambrolizumab, mutational load shows a strong correlation with clinical response. In melanoma patients treated with ipilimumab, long-term benefit is also associated with a higher mutational load, although less significantly. The predicted MHC binding neoantigens in patients with a long-term clinical benefit were enriched for a series of tetrapeptide motifs that were not found in tumors of patients with no or minimal clinical benefit. However, human neoantigens identified in other studies do not show the bias toward tetrapeptide signatures.

As used herein, the terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (γδ T cells), and the like. Additional types of helper T cells include cells such as Th3, Th17, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

As used herein, the term “naive T cell” or Tn, refers to mature T cells that, unlike activated or memory T cells, have not encountered their cognate antigen within the periphery. Naive T cells are commonly characterized by the surface expression of L-selectin (CD62L); the absence of the activation markers CD25, CD44 or CD69; and the absence of the memory CD45RO isoform. They also express functional IL-7 receptors, consisting of subunits IL-7 receptor-a, CD 127, and common-γ chain, CD 132. In the naive state, T cells are thought to be quiescent and non-dividing, requiring the common-gamma chain cytokines IL-7 and IL-15 for homeostatic survival mechanisms.

As used herein, the term “NK cell” or “Natural Killer cell” refer to a subset of peripheral blood lymphocytes defined by the expression of CD56 or CD16 and the absence of the T cell receptor (CD3).

As used herein, the term “central memory T cells” or Tcm, refers to a subgroup or subpopulation of T cells that have lower expression or pro-apoptotic signalling genes, for example, Bid, Bnip3 and Bad, and have higher expression of genes associated with trafficking to secondary lymphoid organs, which genes include CD62L, CXCR3, CCR7, in comparison to effector memory T cells, or Tern.

As used herein, the term “stem memory T cells,” or “stem cell memory T cells”, or Tscm, refers to a subgroup or subpopulation of T cells that are capable of self-renewing and generating Tcm, Tern and Teff (effector T cells), and express CD27 and lymphoid homing molecules such as CCR7 and CD62L, which are properties important for mediating long-term immunity. As used herein, the term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3- and CD56+, expressing and have at least one of CD57+, NKG2C and CD57, and optionally, CD16, but lack expression of one or more of the following: +, low PLZF, low SYK, FceRy, and low FcsRy, low EAT-2., low TIGIT, low PD1, low CD7, low CD161, high LILRB1, high CD45RO, and low CD45RA. In some embodiments, isolated subpopulations of CD56+NK cells comprise expression of NKG2C and CD57. In some other embodiments, isolated subpopulations of CD56+NK cells comprise expression of CD57, CD16, NKG2C, CD57, NKG2D, NCR ligands, NKp30, NKp40, NKp46, activating and inhibitory KIRs, NKG2A and/or DNAM-1. CD56+ can be dim or bright expression.

As used herein, the term “NKT cells” or “natural killer T cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are currently recognized. Invariant or type I NKT cells express a very limited TCR repertoire—a canonical a-chain (Va24-Jal 8 in humans) associated with a limited spectrum of β chains (ΛiβI 1 in humans). The second population of NKT cells, called nonclassical or noninvariant type II NKT cells, display a more heterogeneous TCR αβ usage. Type I NKT cells are currently considered suitable for immunotherapy. Adaptive or invariant (type I) NKT cells can be identified with the expression of at least one or more of the following markers, TCR Va24-Jal 8, Vb11, CD1 d, CD3, CD4, CD8, aGalCer, CD 161 and CD56.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, tissue, biopsy. The term also includes a cell that is removed from at least one, some or all components as the cell is found in non-naturally occurring environments, for example, culture, cell suspension. Therefore, an isolated cell is partly or completely separated from at least one component, including other substances, cells or cell populations, as it is found in nature or as it is grown, stored or subsisted in non-naturally occurring environments. Specific examples of isolated cells include partially pure cells, substantially pure cells and cells cultured in a medium that is non-naturally occurring. Isolated cells may be obtained from separating the desired cells, or populations thereof, from other substances or cells in the environment, or from removing one or more other cell populations or subpopulations from the environment. As used herein, the term “purify” or the like refers to increasing purity. For example, the purity can be increased to at least 50%, 60%, 70%, 80%, 90%, 95%, 99%, or 100%.

As used herein, the term “population” when used with reference to T, NK or NKT cells refers to a group of cells including two or more T, NK, or NKT cells, respectively. Using T cell as an example, the isolated, or enriched, population of T cells may include only one type of T cell, or may include a mixture of two or more types of T cell. The isolated population of T cells can be a homogeneous population of one type of T cell or a heterogeneous population of two or more types of T cell. The isolated population of T cells can also be a heterogeneous population having T cells and at least a cell other than a T cell, e.g., a B cell, a macrophage, a neutrophil, an erythrocyte, a hepatocyte, an endothelial cell, an epithelial cell, a muscle cell, a brain cell, etc. The heterogeneous population can have from 0.01% to about 100% T cell. Accordingly, an isolated population of T cells can have at least 50%, 60%, 70%, 80%, 90%, 95%, 98%, or 99% T cells. The isolated population of T cells can include one or more, or all of, the different types of T cells, including but not limited to those disclosed herein. In an isolated population of T cells that includes more than one type of T cells, the ratio of each type of T cell can range from 0.01% to 99.99%. The isolated population also can be a clonal population of T cells, in which all the T cells of the population are clones of a single T cell.

An isolated population of T, NK or NKT cells may be obtained from a natural source, such as human peripheral blood or cord blood. Various ways of dissociating cells from tissues or cell mixtures to separate the various cell types have been developed in the art. In some cases, these manipulations result in a relatively homogeneous population of cells. The T cells can be isolated by a sorting or selection process as described herein or by other methods known in the art. The proportion of T cells in the isolated population may be higher than the proportion of T cells in the natural source by at least about 10%, about 20%, about 30%, about 40%, about 50%, about 60%, about 70%, about 80%, about 85%, about 90%, or about 95%. The isolated population of T cells can be for T cells in general, or one or more specific types of T cells.

As used herein, the term “subpopulation” when used in reference to T, NK or NKT cells refers to a population of T, NK or NKT cells that includes less than all types of T, NK, or NKT cells, respectively, that are found in nature.

Agents for Improving Efficacy of Cell-Based Adoptive Immunotherapy

The present invention provides a composition comprising one or more glycosylation inhibitors in an amount sufficient for improving therapeutic potential of immune cells suitable for adoptive cell-based therapies Immune cells having improved therapeutic potential present improved proliferation, persistence, cytotoxicity, and/or cell recall/memory Immune cells may have specifically improved in vivo proliferation, in vivo persistence, in vivo cytotoxicity, and/or in vivo cell recall/memory. To improve immune cell therapeutic potential generally requires better quality of the immune cells—in a T cell population, for example, increased number or ratio of naive T cell, stem cell memory T cell, and/or central memory T cell through maintenance, expansion, differentiation, and/or de-differentiation thereof are indicative of better quality of the T cells for improved in vivo adoptive therapeutic potential. In a NK cell population, for example, increased number or ratio of adaptive NK cells through maintenance, subtype skewing, expansion, differentiation, and/or de-differentiation thereof are indicative of better quality of the NK cells for improved in vivo adoptive therapeutic potential. With respect to a NKT cell population, for example, an increased number or ratio of type I NKT cells through maintenance, subtype switching, expansion, differentiation, and/or de-differentiation thereof are indicative of better quality of the NKT cells for improved in vivo adoptive therapeutic potential.

The immune cells suitable for adoptive cell-based therapies are contacted, treated, or modulated with one or more glycosylation inhibitors as defined above. The treatment with the agent(s) can modify the biological properties of the cells, or a subpopulation of the cells, including by modulating cell expansion, maintenance, differentiation, dedifferentiation, and/or survival rate, and/or increasing proliferation, cytotoxicity, persistence, and/or cell recall/memory, and thus the therapeutic potential of the cells treated. For example, the treatment can improve the therapeutic immune cell survival rate both in vitro and in vivo. Further, the treatment can alter the ratios of different subpopulation of the treated cell population. For example, in one embodiment, the number and proportion of naive T cells, stem cell memory T cells, and/or central memory T cells increase in an isolated T cell population upon treatment using one or more of the glycosylation inhibitors of the invention as defined above, and derivatives and analogues thereof. In another embodiment, upon treatment of a NK cell population using one or more of the glycosylation inhibitors of the invention as defined above, and derivatives and analogues thereof, the number and percentage of adaptive NK cells are increased in the population.

Without being limited by the theory, the glycosylation inhibitors of the invention as defined above improve the therapeutic potential of an immune cell for adoptive therapy by modulating cell expansion, metabolism, and/or cell differentiation via regulating cell metabolism, nutrient sensing, proliferation, apoptosis, signal transduction, properties relating to infective process, and/or other aspects of cell function. As understood by those skilled in the art, the scope of the present invention also includes analogues or derivatives, including but not limited to, salt, ester, ether, solvate, hydrate, stereoisomer or prodrug of the listed glycosylation inhibitors of the invention as defined above.

As used herein, the term “synergy” or “synergistic” refers to a combination of two or more entities for an enhanced effect such that the working together of the two or more entities produces an effect greater than the sum of their individual effects, as compared to “antagonistic,” which is used when two or more entities in a combination counteract or neutralize each other's effect; and compared to “additive,” which is used when two or more entities in a combination produce an effect nearly equal to the sum of their individual effects.

The present invention provides a composition comprising an isolated population or subpopulation of immune cells that have been contacted with one or more glycosylation inhibitors of the invention as defined above. In one embodiment, the isolated population or subpopulation of immune cells have been contacted with one or more glycosylation inhibitors of the invention as defined above in an amount sufficient to improve the therapeutic potential of the immune cells. In some embodiments, the treated immune cells are used in a cell-based adoptive therapy. The present invention further provides a population or subpopulation of immune cells, and one or more glycosylation inhibitors selected from the glycosylation inhibitors of the invention as defined above, wherein a treatment of the population or subpopulation of immune cells using the one or more glycosylation inhibitors selected from the glycosylation inhibitors of the invention as defined above improves the therapeutic potential of the immune cells for adoptive therapy. The treatment can modify the biological properties of the immune cells to improve cell proliferation, cytotoxicity, and persistence, and/or reduce the relapse rate of the cell therapy.

In some embodiments, the population of immune cells comprises T cells. In some embodiments, the population of immune cells comprises NK cells. In some embodiments, the population of immune cell comprises NKT cell.

In some embodiments, a population or subpopulation of T cells contacted with one or more glycosylation inhibitors of the invention as defined above comprises an increased number or ratio of naive T cells (Tn), stem cell memory T cells (Tscm), and/or central memory T cells (Tcm), and/or improved cell proliferation, cytotoxicity, cell recall, and/or persistence in comparison to the T cells without the same treatment. In some embodiments the number of Tn, Tscm, and/or Tcm is increased by at least 10%, 20%, 30%, 40%, 50%, 60%, 70%, 80%, 90%, or increased by at least 5, 10, 15, or 20 fold compared to the number of Tn, Tscm, and/or Tcm in the cell population without the same treatment with one or more glycosylation inhibitors of the invention as defined above.

In some embodiments, a population or subpopulation of NK cells contacted with one or more glycosylation inhibitors of the invention as defined above comprises an increased number or ratio of adaptive (or memory) NK cells, and/or improved cell proliferation, cytotoxicity, cell recall, and/or persistence in comparison to the NK cells without the same treatment. In some embodiments the number of adaptive NK cells is increased by at least 10%, 20%, 30%, 40%, 50%, 60%, 70%, 80%, 90%, or increased by at least 5, 10, 15, or 20 fold compared to the number of adaptive NK cells in the cell population without the same treatment with one or more glycosylation inhibitors of the invention as defined above.

In some embodiments, the population or subpopulation of T, NK or NKT cells are genomic ally engineered, which include insertion, deletion, or nucleic acid replacement. Modified immune cells may express cytokine transgenes, silenced inhibitory receptors; or overexpress activating receptors, or CARs for retargeting the immune cells. In some embodiments, the population of immune cells isolated for modulation from a subject, or donor, or isolated from or comprised in peripheral blood, bone marrow, lymph node tissue, cord blood, thymus tissue, tissue from a site of infection, ascites, pleural effusion, spleen tissue, tumours of a subject/donor may be genetically modified. In some embodiments, the isolated population of immune cells are genomically engineered and comprise an insertion, a deletion, and/or a nucleic acid replacement. In some particular embodiments, the immune cells comprise an exogenous nucleic acid encoding a T Cell Receptor (TCR), a Chimeric Antigen Receptor (CAR), and/or overexpression of CD 16 or a variant thereof.

The present invention provides a method of modulating a population or a subpopulation of immune cells suitable for adoptive cell-based therapies, and the method comprises contacting the immune cells with a composition comprising at least one agent of the invention as defined above.

In one embodiment, the method of modulating a population or a subpopulation of immune cells suitable for adoptive cell-based therapies comprises contacting the immune cells with a composition comprising at least one glycosylation inhibitor of the invention as defined above wherein the contacted immune cells have increased cell expansion, increased number or ratio of one or more desired cell subpopulations, and/or improved proliferation, cytotoxicity, cell recall, and/or persistence in comparison to immune cells without contacting the glycosylation inhibitors of the invention as defined above.

In some embodiments, the method of modulating a population or a subpopulation of immune cells suitable for adoptive cell-based therapies comprises contacting the immune cells with a composition comprising at least one glycosylation inhibitor of the invention as defined above, wherein the maintenance and expansion of one or more desired subpopulation of cells are improved in comparison to immune cells without contacting the glycosylation inhibitors of the invention as defined above.

In some embodiments, the method of modulating a population or a subpopulation of immune cells suitable for adoptive cell-based therapies comprises contacting the immune cells with a composition comprising at least one agent of the invention as defined above, wherein the number or ratio of immune cells in the population reprogrammed to a desired state of differentiation is increased in comparison to immune cells without contacting the glycosylation inhibitors of the invention as defined above.

In some embodiments, the method of modulating a population or a subpopulation of immune cells suitable for adoptive cell-based therapies comprises contacting the immune cells with a composition comprising at least one glycosylation inhibitors of the invention as defined above in a sufficient amount for increasing cell expansion, increasing number or ratio of one or more desired immune cell subpopulations, and/or improving proliferation, cytotoxicity, cell recall, and/or persistence of the immune cell in comparison to immune cells without contacting the glycosylation inhibitors of the invention as defined above. In one embodiment, the agent for immune cell treatment is between about 0.1 nM to about 50 μM. In one embodiment, the agent for immune cell treatment is about 0.1 nM, 0.5 nM, 1 nM, 5 nM, 10 nM, 50 nM, 100 nM, 500 nM, 1 μM, 5 μM, 10 μM, 20 μM, or 25 μM, or any concentration in-between. In one embodiment, the agent for immune cell treatment is between about 0.1 nM to about 5 nM, is between about 1 nM to about 100 nM, is between about 50 nM to about 250 nM, between about 100 nM to about 500 nM, between about 250 nM to about 1 μM, between about 500 nM to about 5 μM, between about 3 μM to about 10 μM, between about 5 μM to about 15 μM, between about 12 μM to about 20 μM, or between about 18 μM to about 25 μM.

In some embodiments, the method of modulating a population or a subpopulation of immune cells suitable for adoptive cell-based therapies comprises contacting the immune cells with a composition comprising at least one glycosylation inhibitor of the invention as defined above for a sufficient length of time for increasing cell expansion, increasing number or ratio of one or more desired immune cell subpopulations, and/or improving proliferation, cytotoxicity, cell recall, and/or persistence of the immune cell in comparison to immune cells without contacting the glycosylation inhibitors of the invention as defined above. In one embodiment, the immune cells are contacted with one or more glycosylation inhibitor of the invention as defined above for at least 10 minutes, 30 minutes, 1 hours, 2, hours, 5 hours, 12 hours, 16 hours, 18 hours, 1 day, 2 days, 3 days, 4 days, 5 days, 6 days, 7 days, 10 days, 15 days, 20 days, 25 days, 30 days, or any length of period in between. In one embodiment, the immune cells are contacted with one or more glycosylation inhibitor of the invention as defined above for between about 0.5 hour to about 2 hours, between about 1 hour to about 12 hours, between about 10 hours to about 2 days, between about 1 day to about 3 days, between about 2 days to about 5 days, between about 3 days to about 6 days, between about 5 days to about 8 days, between about 7 days to about 14 days, between about 12 days to about 22 days, between about 14 days to about 25 days, between about 20 days to about 30 days. In some embodiments, the immune cells are contacted with one or more glycosylation inhibitor of the invention as defined above for no less than 16 hours, 14 hours, 12 hours, 10 hours, 8 hours, 6 hours, 4 hours, 2 hours, or any length of time in between. As such, said sufficient length of time, for example, is no less than 15, 13, 11, 9, 7, 5, 3, or 1 hour(s). In some other embodiments of the method, said sufficient length of time is no less than 24 hours, 36 hours, 48 hours, 60 hours, 72 hours, or any length of time in between. As such, said sufficient length of time is, for example, no less than 30, 42, 54, 66, 78, 90 hour(s).

The method of modulating a population or a subpopulation of immune cells suitable for adoptive cell-based therapies that comprises contacting the immune cells with a composition comprising at least one glycosylation inhibitor of the invention as defined above, may further comprise enriching or isolating one or more desired subpopulations from the immune cells after the contacting, wherein the one or more desired subpopulations are selected from the group consisting of naive T cell, stem cell memory T cell, central memory T cell, adaptive NK cell, and type I NKT cell.

In some other embodiments, the isolated immune cells for modulation are genetically modified (genetically engineered or naturally derived from rearrangements, mutations, genetic imprinting and/or epigenetic modification). In some embodiments, the isolated immune cells for modulation comprise at least one genetically modified modality. In some embodiments, the isolated population of immune cells are genomically engineered and comprise an insertion, a deletion, and/or a nucleic acid replacement. In some particular embodiments, the immune cells comprise an exogenous nucleic acid encoding a T Cell Receptor (TCR), a Chimeric Antigen Receptor (CAR), and/or overexpression of CD 16 or a variant thereof. As such, the genetically modified immune cells are isolated for ex vivo modulation using the present compositions and methods as disclosed. In some embodiments, after modulation, the genetically modified immune cells isolated from a subject may be administered to the same donor or a different patient. In some embodiments, the donor derived immune cells for modulation comprise an exogenous nucleic acid encoding a T Cell Receptor (TCR) and/or a Chimeric Antigen Receptor (CAR).

The present invention provides a composition comprising an isolated population or subpopulation of immune cells that have been contacted with one or more glycosylation inhibitor of the invention as defined above in an amount sufficient to improve the therapeutic potential of the immune cells when used in a cell based adoptive therapy.

A variety of diseases may be ameliorated by introducing the cells of the invention to a subject suitable for adoptive cell therapy. Examples of diseases including various autoimmune disorders, including but not limited to, alopecia areata, autoimmune hemolytic anemia, autoimmune hepatitis, dermatomyositis, diabetes (type 1), some forms of juvenile idiopathic arthritis, glomerulonephritis, Graves' disease, Guillain-Barre syndrome, idiopathic thrombocytopenic purpura, myasthenia gravis, some forms of myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjogren's syndrome, systemic lupus, erythematosus, some forms of thyroiditis, some forms of uveitis, vitiligo, granulomatosis with poly angiitis (Wegener's); haematological malignancies, including but not limited to, acute and chronic leukaemias, lymphomas, multiple myeloma and myelodysplastic syndromes; solid tumours, including but not limited to, tumour of the brain, prostate, breast, lung, colon, uterus, skin, liver, bone, pancreas, ovary, testes, bladder, kidney, head, neck, stomach, cervix, rectum, larynx, or oesophagus; and infections, including but not limited to, HIV-(human immunodeficiency virus), RSV-(Respiratory Syncytial Virus), EBV-(Epstein-Barr virus), CMV-(cytomegalovirus), HBV, HCV, adenovirus- and BK polyomavirus-associated disorders.

In embodiments of any of the methods and compositions described herein, the cancer is a solid cancer selected from the group consisting of colon cancer, rectal cancer, renal-cell carcinoma, liver cancer, non-small cell carcinoma of the lung, cancer of the small intestine, cancer of the oesophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angio genesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In embodiments of any of the methods and compositions described herein, the cancer is a hematologic cancer chosen from one or more of chronic lymphocytic leukaemia (CLL), acute leukaemias, acute lymphoid leukaemia (ALL), B-cell acute lymphoid leukaemia (B-ALL), T-cell acute lymphoid leukaemia (T-ALL), chronic myelogenous leukaemia (CML), B cell prolymphocytic leukaemia, blastic plasmacytoid dendritic cell neoplasm, Burkitt's lymphoma, diffuse large B cell lymphoma, follicular lymphoma, hairy cell leukaemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia.

The present invention will be illustrated by means of non-limiting examples in reference to the following figures.

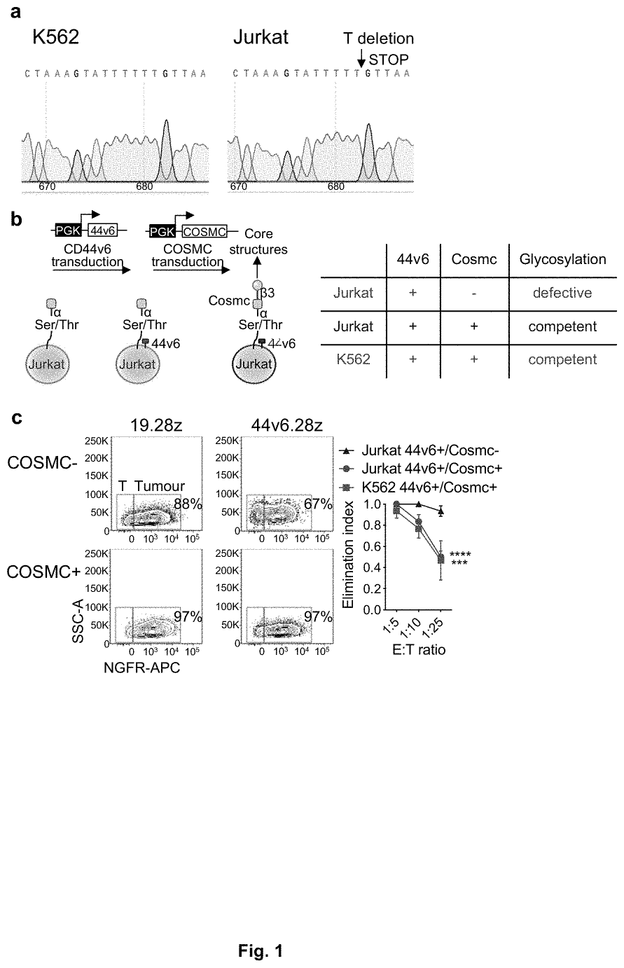

FIG. 1: O-glycosylation on tumour cells impairs recognition by CD44v6 CAR-T cells. a, DNA sequencing of Cosmc amplicons from the mutated Jurkat samples compared to K562 controls. b, Left: schematic representation of the generated Jurkat model cell lines. c, CD44v6 CAR-T cells (44v6.28z) or control CD19 CAR-T cells (19.28z) were cultured with glycosylation competent (44v6+/Cosmc+) or incompetent (44v6+/Cosmc−) Jurkat cells at 1:5, 1:10, 1:25 effector to target ratios (E:T). After 4 days, target cell killing was analysed by FACS and expressed as elimination index [1−(number of residual tumour cells with 44v6.28z CAR-T cells/number of residual tumour cells with 19.28z CAR-T cells)]. Left: FACS plots from a representative donor. Right: data obtained from n=3 donors (means±SEM). Results from a two-way ANOVA are shown when statistically significant (***P<0.001; ****P<0.0001).

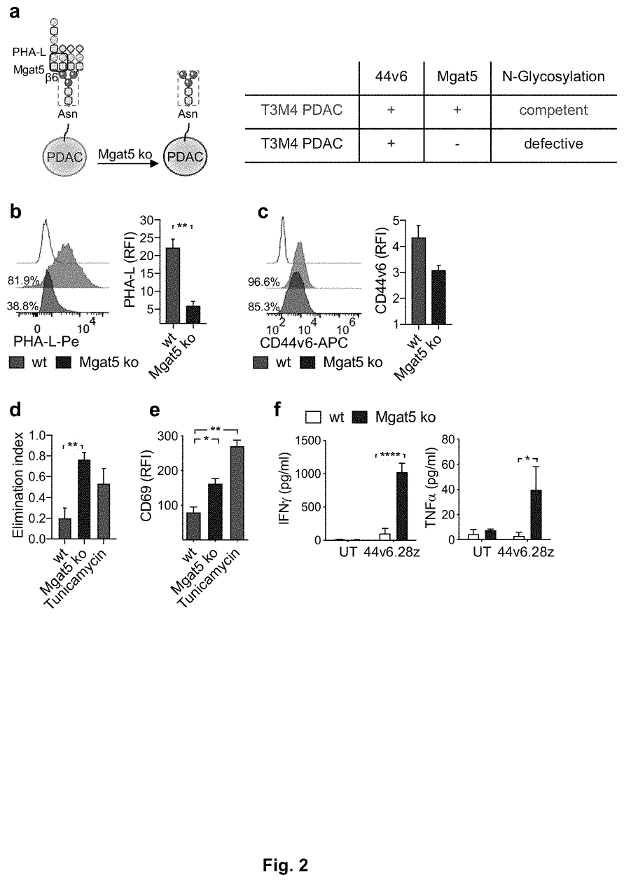

FIG. 2: N-glycosylation on tumour cells impairs recognition by CD44v6 CAR-T cells. N-glycosylation was hampered in T3M4 pancreatic adenocarcinoma cell lines by knocking out the expression of the glycosyltransferase Mgat5 and the glyco-phenotype was assessed by PHA-L staining. a, Schematic representation of the model T3M4 pancreatic adenocarcinoma cell lines generated (PDAC). b,c, Staining of either wt (N-glycosylation competent) or Mgat5 ko (N-glycosylation defective) T3M4 tumours with PHA-L, that binds complex N-glycans, and with the CAR's target antigen CD44v6. d,e, CD44v6 CAR-T cells (44v6.28z) were challenged with either wt or Mgat5 ko or with tunicamycin treated (100 ng/ml, 48 h) T3M4 tumours. After 4 days, killing was expressed as elimination index (d) and T cell activation as CD69 up-regulation (e). A one-way ANOVA was used for statistical analysis (*P<0.05; **P<0.01). f, IFNγ and TNFα production by CD44v6 CAR T cells (44v6.28z) was measured 24 hours after stimulation with either wt or Mgat5 ko pancreatic tumour cells. A two-way ANOVA was used for statistical analysis (*P<0.05; **P<0.01; ****P<0.0001).

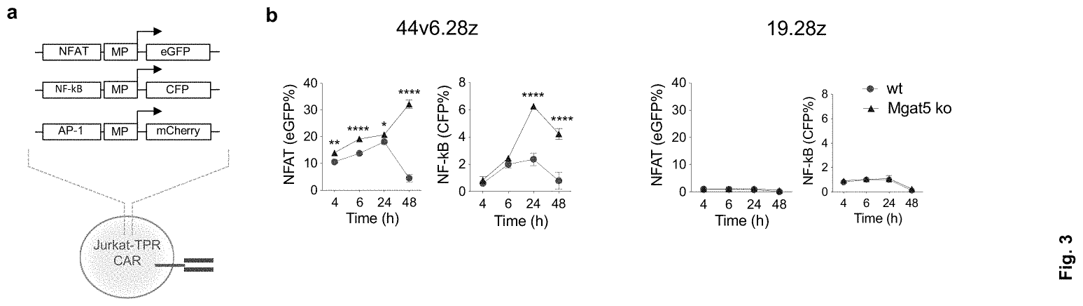

FIG. 3: Mgat5 ko tumours induce a stronger NFAT and NF-kB activation in effector cells. Jurkat triple reporter cells were transduced with CAR T constructs and stimulated with wt (N-glycosylation competent) or Mgat5 ko (N-glycosylation deficient) T3M4 pancreatic tumour cell lines. a, Schematic representation of the Jurkat-CAR⁺ triple reporter (TPR) cell lines generated. b, Time-course activation of Jurkat-CAR+ reporter cells. NFAT and NF-kB activations were assessed by measuring the percentage of eGFP and CFP signals, respectively. A two-way ANOVA was used for statistical analysis (*P<0.05; **P<0.01; ****P<0.0001).

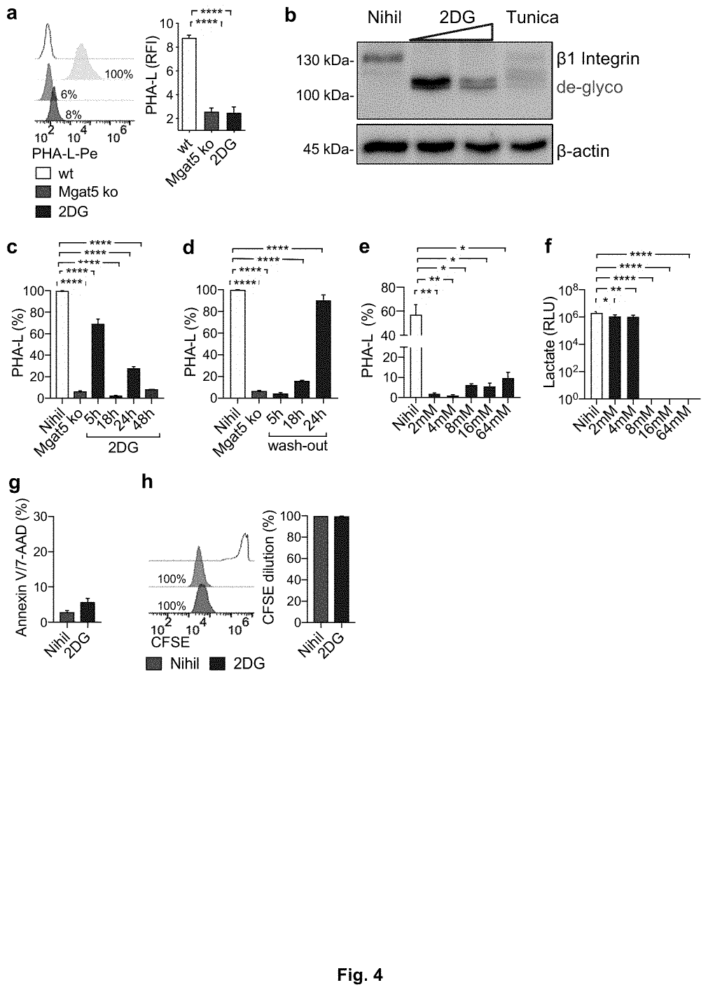

FIG. 4: 2DG inhibits N-glycosylation in pancreatic tumour cells without having direct effects on their survival and proliferation. a, b, T3M4 pancreatic tumour cells were treated 4 mM of 2DG for 48 hours before analysing the glycosylation status compared to Mgat5 ko (N-glycosylation deficient) cells. a, PHA-L staining showing the glyco-phenotype of 2DG treated cells compared to control Mgat5 ko cells. A one-way ANOVA was used for statistical analysis (****P<0.0001). b, Analysis of (31 integrin and CD44v6 glycosylation in total cell lysates of tumours either untreated (nihil) or treated with incremental doses of 2DG (4 mM or 16 mM) or with control tunicamycin (Tunica, 100 ng/ml) for 48 hours. c,d, Tumour cells were treated with 4 mM 2DG for up to 48 hours before treatment wash-out. Kinetics of tumour de-glycosylation (c) and glycosylation (d) were assessed by PHA-L staining. A one-way ANOVA was used for statistical analysis (****P<0.0001). e,f, Dose-response of tumour cells treated with incremental concentrations of 2DG for 48h. N-glycosylation impairment was determined by PHA-L staining (e) and glycolysis tested by lactate production (f). A two-way ANOVA was used for statistical analysis (*P<0.05; **P<0.01; ****P<0.0001). Tumour survival (g) and proliferation (h) was analysed after treatment with 4 mM 2DG for 48 hours and expressed as percentage of 7AADpos cells and percentage of CFSE dilution, respectively.

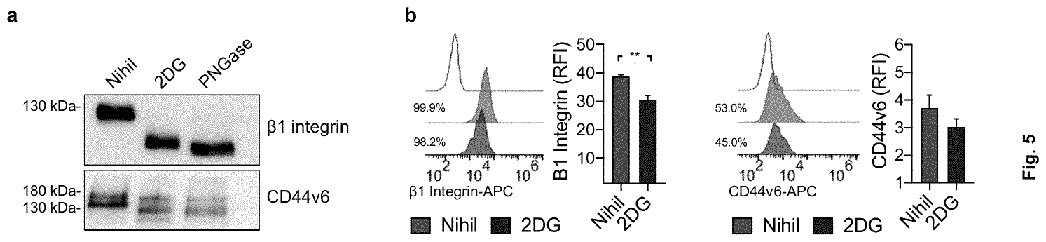

FIG. 5: Blocking glycosylation with low doses of 2DG doesn't impair surface antigen expression. T3M4 pancreatic tumour cells were treated 4 mM of 2DG for 48 hours before analysing antigen expression and cell fitness. a, Biotin-enrichment assay of extracellular de-glycosylated (31 integrin and CD44v6 upon treatment with 2DG or with control PNGase, which cleaves N-glycans. Untreated tumour cells (nihil) are shown as control. b, Extracellular expression of (31 integrin and CD44v6 in tumours either untreated or treated with 4 mM 2DG for 48 hours.

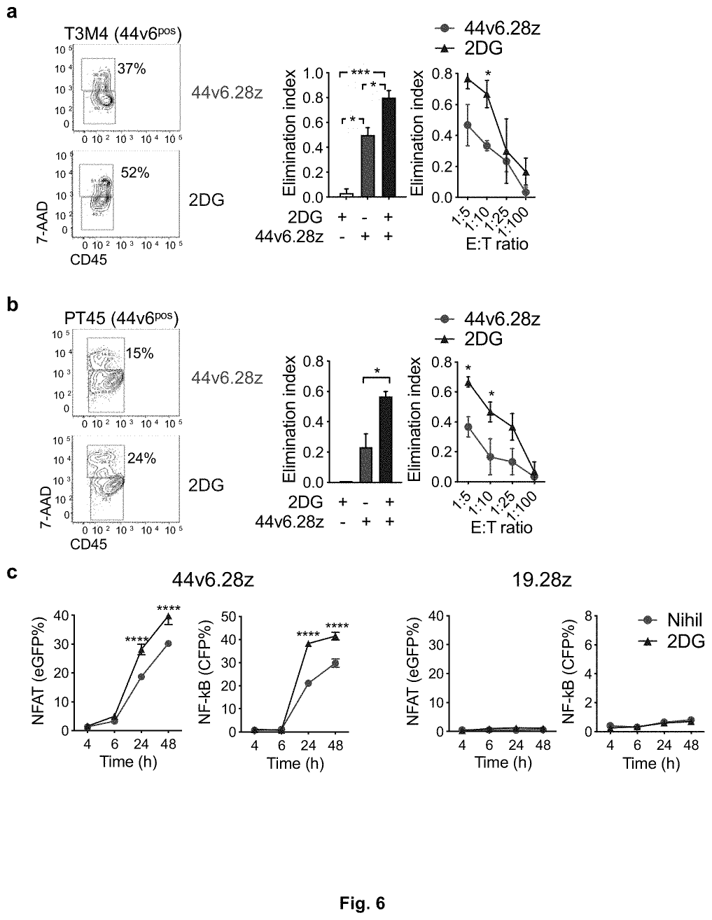

FIG. 6: 2DG increases tumour recognition and killing by CD44v6 CAR-T cells. (a,b) CD44v6^{pos} T3M4 (a) and PT45 pancreatic tumour cells (b) were exposed to 4 mM 2DG for 48 hours before being co-cultured with either CD44v6 CAR-T cells (44v6.28z) or control CD19 CAR-T cells (19.28z). The day after, tumour cells killing was analysed by FACS and expressed as elimination index. Left: FACS plots from a representative donor. Middle: elimination indexes at the 1:10 effector to target ratio (E:T ratio). Right: data obtained from n=3 donors (means±SEM). Results from a 1-way ANOVA are indicated when statistically significant (*, P<0.05; ***, P<0.001). c, Time-course activation of Jurkat-CAR+ reporter cells stimulated with either untreated (nihil) T3M4 tumours or treated with 4 mM 2DG for 48 hours. NFAT and NF-kB activations were assessed by measuring the percentage of eGFP and CFP signals, respectively. A two-way ANOVA was used for statistical analysis (****P<0.0001).

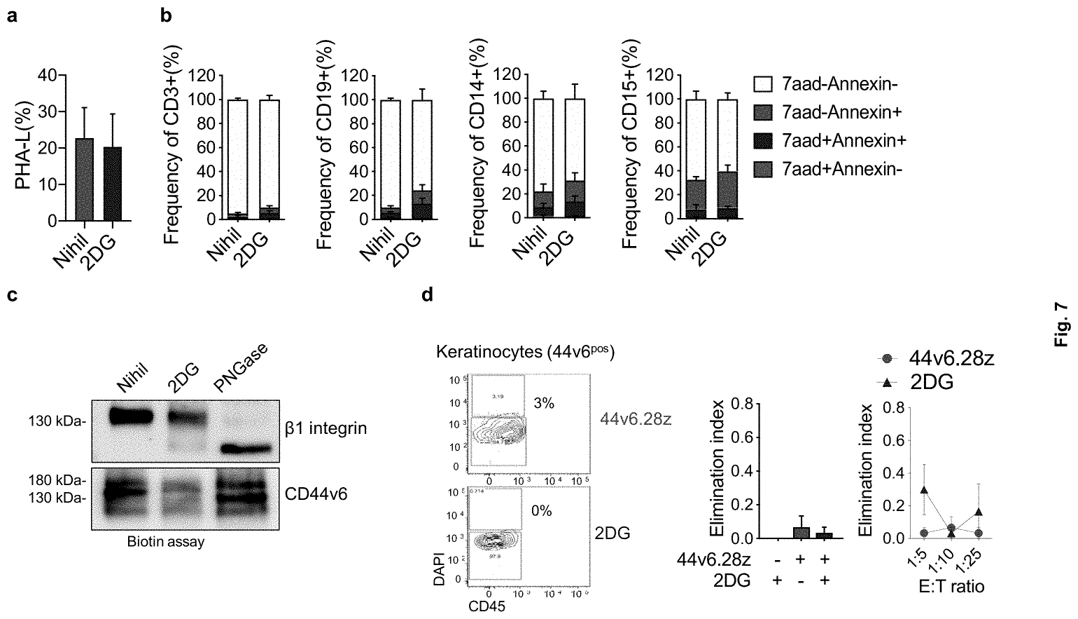

FIG. 7: 2DG doesn't de-glycosylate nor increases killing of healthy cells. a, b, Fresh buffy coat cells were exposed to either medium alone (nihil) or to 4 mM 2DG for 18 hours before analysing cell glycosylation by PHA-L staining (a) and viability by 7aad-AnnexinV staining (b). c,d, Primary keratinocytes were exposed to 2DG for 48 hours before being analysed by western blot or co-cultured with CD44v6 CAR− T cells (44v6.28z). c, Biotin-enrichment assay of extracellular 131 integrin and CD44v6 in primary keratinocytes either untreated (nihil) or treated with 2DG or with control PNGase, which cleaves N-glycosylation sites. d, In vitro killing of primary keratinocytes, either untreated or treated with 2DG, by CD44v6 CAR T cells (44v6.28z). Killing was expressed as elimination index compared to control CD19 CAR-T cells (19.28z).

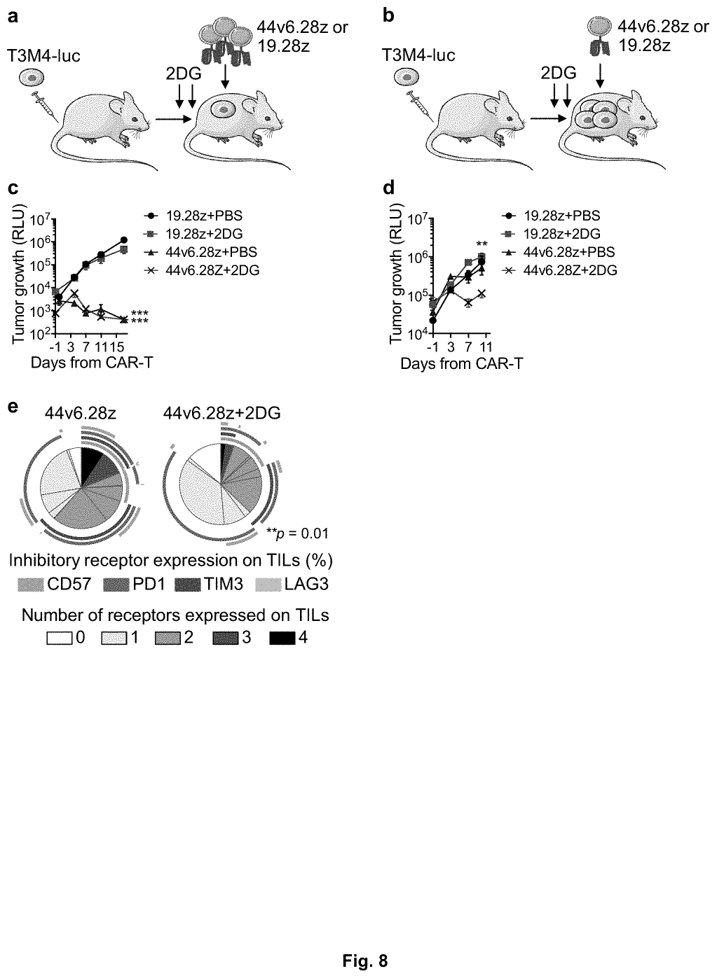

FIG. 8: 2DG increases recognition and killing of pancreatic tumour by CD44v6 CAR-T cells in vivo. NSG mice were injected intra-pancreas with 0,1×10⁶ T3M4 adenocarcinoma cells expressing a secreted luciferase. a,c, On day 2 and 3 from tumour engraftment, when the tumour burden was low, mice were injected intraperitoneally (i.p.) with 500 mg/kg 2DG. At day 3 mice were infused intravenously (i.v.) with 5×10⁵ of either CD44v6 (44v6.28z) or CD19 (19.28z) CAR T cells as control. Tumour burden was monitored by measuring secreted luciferase and a two-way ANOVA was used for statistical analysis (**P<0.01; ***P<0.001). b,d, On day 6 and 7 from tumour engraftment, when the tumour burden was high, mice were injected intraperitoneally (i.p.) with 500 mg/kg 2DG. At day 7 mice were infused intravenously (i.v.) with 5×10⁵ of either CD44v6 (44v6.28z) or CD19 (19.28z) CART cells as control. Tumour burden was monitored by measuring secreted luciferase and a two-way ANOVA was used for statistical analysis (**P<0.01)

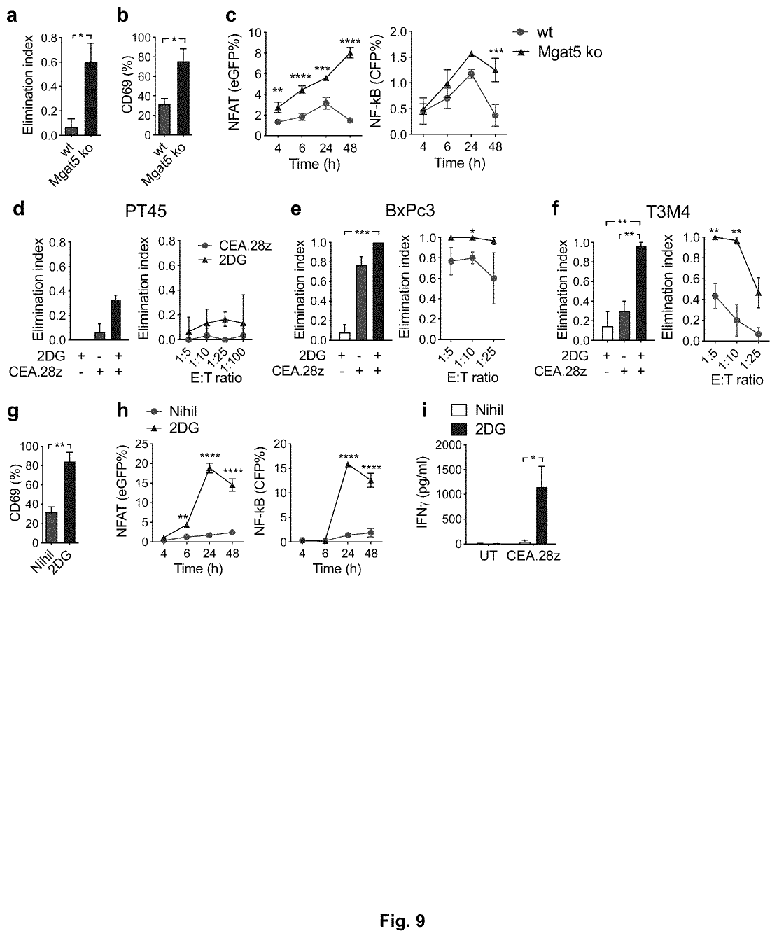

FIG. 9: 2DG increases tumour recognition and killing by CEA CAR-T cells. a,b, CEA CAR-T cells (CEA.28z) or control CD19 CAR T cells (19.28z) were challenged with either wt (N-glycosylation competent) or Mgat5 ko (N-glycosylation deficient) T3M4 pancreatic tumour cells. After 4 days, tumour killing was analysed by FACS and expressed as elimination index (a) and T cell activation as CD69 up-regulation (b). A t-test was used for statistical analysis (*P<0.05). c, Time-course activation of Jurkat-CAR+ reporter cells stimulated with either untreated (nihil) or 2DG-treated T3M4 pancreatic tumours. NFAT and NF-kB activations were assessed by measuring the percentage of eGFP and CFP signals, respectively. A two-way

ANOVA was used for statistical analysis (**P<0.01; ***P<0.001; ****P<0.0001). d-f, CEA⁻ PT45 (d), CEA⁺ BxPc3 (e) and T3M4 (f) cells were exposed to 4 mM 2DG for 48 hours before being co-cultured with CEA CAR-T cells (CEA.28z) or with control CD19 CAR-T cells (19.28z). After 4 days, tumour killing was analysed by FACS and expressed as elimination index. Left: elimination indexes at the 1:10 E:T ratio. Right: data obtained from n=3 donors (means±SEM). Results from a 1-way ANOVA are indicated when statistically significant (*, P<0.05; **, P<0.01; ***, P<0.001). g, Activation of CEA CAR T cells (CEA.28z) co-cultured with either untreated T3M4 pancreatic tumour (nihil) or treated with 4 mM 2DG was measured at 96 hours by CD69 up-regulation. A t-test was used for statistical analysis (**, P<0.01) h, Time-course activation of Jurkat-CAR+ reporter cells stimulated with either untreated T3M4 pancreatic tumour cells (nihil) or treated with 4 mM 2DG for 48 hours. i, IFNγ production by CEA CAR T cells (CEA.28z) was measured 24 hours after stimulation with either untreated (nihil) or 2DG treated pancreatic tumour cells. A two-way ANOVA was used for statistical analysis (*P<0.05).

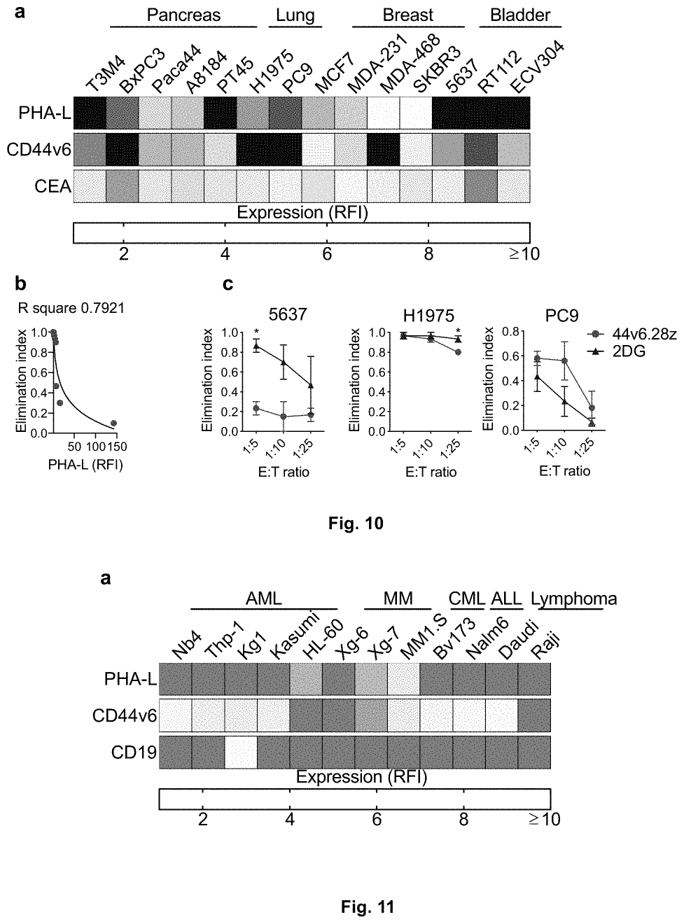

FIG. 10: Complex N-glycans are expressed by several epithelial carcinomas. a, heat map of PHA-L, CD44v6 and CEA expression on cell lines deriving from tumours of the pancreas, lung, breast and bladder. b, Correlation analysis of the expression of PHA-L (left) or CD44v6 (right) and the in vitro tumour killing at the 1:10 E:T ratio by 44v6.28z CAR T cells. c, CD44v6+5637, H1975 and PC9 cells were exposed to 4 mM 2DG for 48 hours before being co-cultured with CD44v6.28z CAR-T cells or control 19.28z CAR-T cells. Tumour killing was analysed by FACS and expressed as elimination index. Results from a two-way ANOVA are shown when statistically significant (*P<0.05).

FIG. 11: Complex N-glycans are expressed by several haematological tumours. a, heat map of PHA-L, CD44v6 and CD19 expression on cell lines deriving from acute myeloid leukaemia (AML), multiple myeloma (MM), chronic myelogenous leukaemia (CML), acute lymphoblastic leukaemia (ALL) and lymphoma.

### DETAILED DESCRIPTION OF THE INVENTION

Materials and Methods

Transduction and Culture Conditions

Activation of T cells from healthy donors was performed with anti-CD3/CD28 immune-conjugated magnetic beads (bCD3/CD28) (ClinExvivo CD3/CD28; Invitrogen) following manufacturer's instructions. T cells were RV-transduced by 2 rounds of spinoculation or LV-transduced by overnight incubation and cultured in RPMI 1640 (Gibco-Brl), fetal bovine serum (10%, BioWhittaker) with IL-7 and IL-15 (5 ng/mL; Peprotech). CAR transduction efficiency and sorting was performed by protein-L (Thermo Scientific) staining according to manufacturer's instructions. Phenotypic analysis and functional testing were performed at day 21 after stimulation. Tumour cells were LV-transduced by overnight incubation and FACS-sorted with marker genes. Haematological tumours, bladder cancer and lung cancer cells were cultured in RPMI 1640 (Gibco-Brl) whereas pancreatic tumour cells were cultured in IMDM (Gibco-Brl). All media for T cell and tumour cell growth were supplemented with penicillin (100 UI/ml; Pharmacia), streptomycin (100 UI/ml; Bristol-Meyers Squibb), glutamine (2 mM; Gibco) and fetal bovine serum (10%, BioWhittaker). XG-6 and XG-7 cells were cultured with addition of IL-6 (2 ng/ml, Peprotech). Primary keratinocytes were purchased from Lonza (L0192627) and cultured in Epi-Life (Invitrogen, M-EPI-500-CA) with the human keratinocytes growth supplement (Invitrogen, S-001-5) following manufacturer's instructions.

Cosmc PCR and Sequencing

Total genomic DNA from Jurkat and K562 cells was isolated using the QIAamp DNA mini kit (Qiagen). For Cosmc gene PCR, the forward primer was 5′-CTCCATAGAGGAGTTGTTGC-3′ (SEQ ID NO. 1) and the reverse primer was 5′-TCACGCTTTTCTACCACTTC-3′ (SEQ ID NO. 2). The expected 1,218-bp PCR band product was analysed on agarose gel, extracted and sequenced.

Retroviral and Lentiviral Constructs

To generate the Cosmc lentiviral (LV) expression vector, DNA sequence was synthetized by GeneArt (ThermoFisher) and cloned into a self-inactivating LV vector with a PGK bidirectional promoter driving the co-expression of CD44v6 or Cosmc molecules and marker genes NGFR or eGFP, respectively. The inventors generated CD44v6, CEA, CD19 CAR constructs by cloning specific scFvs CD44v6, BIWA8 mAb; CEA, BW431-26 mAb; CD19, FMC63 mAb) in a CAR backbone carrying an IgG1-derived hinge spacer, a CD28 costimulatory endodomain and the TCR zeta chain. All CAR constructs were expressed into viral vectors and the supernatants produced in 293T cells. Retroviral vectors were employed as described in Casucci M et al., Blood. 2013 Nov. 14; 122(20):3392 while lentiviral vectors as described in Amendola M et al., Nat Biotechnol 2005 January; 23(1):108-16. Both, the CD44v6 lentiviral expression vector and the secreted luciferase expression vector were employed as previously described (Norelli et al., Nat Med 2018, 24, pages739-748; Bondanza and Casucci, Methods in molecular biology 1393, Tumor immunology methods and protocols, chapter 10).

All sequences generated and cloned into viral vector backbones are listed below.

COSMC sequence (SEQ ID NO. 3):
MLSESSSFLKGVMLGSIFCALITMLGHIRIGHGNRMHHHEHHHLQAPNK
EDILKISEDERMELSKSFRVYCIILVKPKDVSLWAAVKETWTKHCDKAE
FFSSENVKVFESINMDTNDMWLMMRKAYKYAFDKYRDQYNWFFLARPTT
FAIIENLKYFLLKKDPSQPFYLGHTIKSGDLEYVGMEGGIVLSVESMKR
LNSLLNIPEKCPEQGGMIWKISEDKQLAVCLKYAGVFAENAEDADGKDV
FNTKSVGLSIKEAMTYHPNQVVEGCCSDMAVTFNGLTPNQMHVMMYGVY
RLRAFGHIFNDALVFLPPNGSDND
CD19-scFv (FMC63) sequence (SEQ ID NO. 4):
MEFGLSWLFLVAILKGVQCSRDIQMTQTTSSLSASLGDRVTISCRASQD
ISKYLNWYQQKPDGTVKLLIYHTSRLHSGVPSRFSGSGSGTDYSLTISN
LEQEDIATYFCQQGNTLPYTFGGGTKLELKRGGGGSGGGGSGGGGSGGG
GSEVQLQQSGPGLVAPSQSLSVTCTVSGVSLPDYGVSWIRQPPRKGLEW
LGVIWGSETTYYNSALKSRLTIIKDNSKSQVFLKMNSLQTDDTAIYYCA
KHYYYGGSYAMDYWGQGTTVTVSSYVTVSS
CD44v6-scFv (Biwa-8) sequence (SEQ ID NO. 5):
MEAPAQLLFLLLLWLPDTTGEIVLTQSPATLSLSPGERATLSCSASSSI
NYIYWLQQKPGQAPRILIYLTSNLASGVPARFSGSGSGTDFTLTISSLE
PEDFAVYYCLQWSSNPLTFGGGTKVEIKRGGGGSGGGGSEVQLVESGGG
LVKPGGSLRLSCAASGFTFSSYDMSWVRQAPGKGLEWVSTISSGGSYTY
YLDSIKGRFTISRDNAKNSLYLQMNSLRAEDTAVYYCARQGLDYWGRGT
LVTVSS
CEA-scFv (BW431-26) sequence (SEQ ID NO. 6):
MDFQVQIFSFLLISASVIMSRGVHSQVQLQESGPGLVRPSQTLSLTCTV
SGFTISSGYSWHWVRQPPGRGLEWIGYIQYSGITNYNPSLKSRVTMLVD
TSKNQFSLRLSSVTAADTAVYYCAREDYDYHWYFDVWGQGTTVTVSSGG
GGSGGGGSGGGGSDIQLTQSPSSLSASVGDRVTITCSTSSSVSYMHWYQ
QKPGKAPKLLIYSTSNLASGVPSRFSGSGSGTDFTFTISSLQPEDIATY
YCHQWSSYPTFGQGTKVEIKV
Hinge sequence (SEQ ID NO. 7):
EPKSCDKTHTCPPCP
CD28 sequence (SEQ ID NO. 8):
FWVLVVVGGVLACYSLLVTVAFIIFWVRSKRSRLLHSDYMNMTPRRPGP
TRKHYQPYAPPRDFAAYRS
CD3ζ chain sequence (SEQ ID NO. 9):
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR*

2DG Treatment

For dose assessment, tumour cells were exposed to 2, 4, 8, 16 or 64 mM 2DG (D8375, Sigma-Aldrich) and, after 48h, absolute cell numbers and percentages of necrotic and proliferating cells were determined by FACS using Flow-Count Fluorospheres (BeckmanCoulter), 7-AAD and CFSE, respectively. For pharmacokinetic studies, tumour cells were treated with 4 mM 2DG and surface N-glycosylation was assessed by PHA-L staining at different time points from treatment or from wash-out to assess the kinetic of de-glycosylation and glycosylation, respectively. For safety studies, buffy coat cells were treated with 4 mM 2DG for 18 hours in the presence of IL-2 (100 IU/mL; Chiron), IL-21 (10 ng/ml; Peprotech) and IL-15 (5 ng/mL; Peprotech) before assessment of the glyco-phenotype by PHA-L staining and of cell viability by 7aad/AnnexinV staining.

In Vitro Co-Culture Assay

CEA or CD44v6 CAR-T cells from healthy donors were co-cultured at 1:5, 1:10, 1:25 E:T ratios with target cells in the absence of IL-7 and IL-15. T cells transduced with an irrelevant CAR (CD19 CAR T) were used as control. After 24h or 96h, co-cultures were analysed by FACS using Flow-Count Fluorospheres (BeckmanCoulter) and target cell killing, expressed as Elimination Index, was calculated as follows: 1−(number of residual target cells with experimental CAR-T cells/number of residual target with irrelevant CAR-T cells). In co-culture assays combining CAR-T cells with 2DG, target cells were exposed to 4 mM 2DG for 48h before being washed, co-cultured with experimental or irrelevant CAR-T cells and analysed as above. In these experiments, the Elimination Index of 2DG alone was calculated as follows: 1-(number of target cells cultivated in medium supplemented with 2DG/number of target cells cultivated in medium alone). Co-culture of Jurkat CAR-TPR and tumour cells was performed at 1:1 E:T ratio. As control, Jurkat CAR-TPR cells were treated with either 50 ng/mL of phorbol myristate acetate (PMA) or 1 μg/mL of lonomycin or a combination of the two. Upregulation of fluorescence was assessed after 24 hours

Flow Cytometry

The inventors used mAbs specific for human CD44v6 (e-Bioscience), CD3, CD45, NGFR, LAG3 (BD Bioscience), HLA-DR, PD1, TIM3, (Biolegend), CD57 (Miltenyi Biotec). To determine cell vitality, 7-Aminoactinomicin D (7-AAD), AnnexinV or DAPI reagents were used. For LV transduced tumour cells, GFP expression was analysed by direct fluorescence. Samples were run through a fluorescence-activated cell sorting (FACS) Canto flow cytometer (BD Biosciences) and data were analysed with the FlowJo software (Tree Star, Inc.). For branched N-glycans expression analysis, cells were incubated with biotinylated Phaseolus vulgaris Leukoagglutinin (PHA-L; Caderlane) for 1 hour according to manufacturer's instructions, washed and incubated with PE-conjugated streptavidin and analysed by flow cytometry.

Generation of CAR⁺ Jurkat Triple Reporter Cells

The triple reporter Jurkat T cell line (Jurkat TPR) was kindly provided by the group of Steinberger (S. Jutz et al., Journal of Immunological Methods 430 (2016) 10-20). First, cells were transduced by overnight incubation with lentiviral vectors expressing CAR constructs, either specific for CD44v6 (44v6.28z) or CD19 (19.28z). Next, transduction efficiency was checked by flow cytometry after a week by looking at the percentage of cells positive for the marker gene NGFR. Finally, CAR′ Jurkat TPR cells were co-cultured with target cells at the effector to target (E:T) ratio of 1:1 and reporter gene activation was assessed at 4, 6, 24 or 48 hours by flow cytometry using CytoFLEX (Beckman Coulter).

Generation of Mgat5 Knocked-Out Pancreatic Tumour Cells

The lentiviral vector plasmid encoding for Mgat5-specific gRNA and Cas9 protein was purchased from ABM good (K1298706). After lentiviral vector production, T3M4 pancreatic tumour cell line was transduced and cultivated in puromycin supplemented medium for positive selection according to manufacturing instructions (Thermo Fisher, A1113803).

Western Blot Assays

Tumour cells or healthy primary keratinocytes were treated with either medium alone or supplemented with 4 mM 2DG for 48h, lysed and subjected to SDS polyacrylamide gel electrophoresys. De-glycosylation was assessed as molecular weight shift. For selective cell membrane protein analysis, biotinilation assay was performed according to manufacturer instructions (Thermo Scientific). Treatment of cell lysates with PNGase F (NEB, P0704) was performed as recommended by the manufacturer's instructions.

In Vivo Efficacy Experiment

All experiments were approved by the Institutional Animal Care and Use Committee (IACUC) of San Raffaele University Hospital and Scientific Institute and by the Italian Governmental Health Institute (Rome, Italy). NSG mice (NOD.Cg-Prkdcscid Il2rgtm1Wj1) were obtained from the Jackson Laboratories and kept in a specific-pathogen-free (SPF) facility within individually ventilated cages. 9-week-old NSG mice were injected intra-pancreas with 0,1×10⁶ T3M4 tumour cells expressing a secreted luciferase (Bondanza and Casucci, Methods in molecular biology 1393, Tumor immunology methods and protocols, chapter 10). At day 6 and 7 from tumour engraftment, when the tumour burden was high, mice were injected intraperitoneally (i.p.) with 500 mg/kg 2DG. At day 7 mice were infused intravenously (i.v.) with 5×10⁵ of either CD44v6 or CD19 CAR T cells as control. In another set of experiments, mice were treated with 500 mg/kg 2DG at day 2 and 3 and received 10×10⁵ of either CD44v6 or CD19 CAR T cells at day 3, when the tumour burden was low. Tumour growth was monitored by bioluminescence assay using the QUANTI-Luc detection reagent (InvivoGen) and expressed as relative light units (RLUs), according to the manufacturer instructions. Mice were sacrificed when RLUs were >10⁶. At sacrifice, tumour masses were retrieved, dissociated using gentle MACS (Miltenyi Biotec) and tumour dissociation reagents (Miltenyi Biotec, 130-095-929) following manufacturer's instructions and analysed by FACS.

Lactate Production Assay

Lactate production was assessed after 48 hours of 2DG treatment at the doses of 2, 4, 8, 16 or 64 mM 2DG using the Lactate-Glo assay (Promega) according to the manufacturer's instructions.

Statistical Analysis

Statistical analysis was performed using Graphpad Prism 5.0a software version. All data are presented as mean+/−SEM. T-test, One-way or Two-way ANOVA was used to determine the statistical significance of differences between samples. Differences with a P value<0.05 were considered statistically significant.

### Examples

### Example 1: Glycosylation Protects CD44v6+ Tumour Cells from CD44v6 CAR-T Cell Killing

The inventors recently developed and optimized a CD44v6 CAR able to tackle multiple tumour types including acute myeloid leukaemia, multiple myeloma and several epithelial carcinomas (Casucci M et al., Blood. 2013 Nov. 14; 122(20):3392-4). Since CD44v6 is extensively glycosylated (Ponta H et al., Nat Rev Mol Cell Biol. 2003 January; 4(1):33-45) and sugar chains may be sterically hulking, the inventors investigated whether glycosylation, either 0- or N-linked, could influence its targeting by CD44v6 CAR-T cells. As a first step to answer this question, the inventors took advantage of the Jurkat T-cell leukaemia cell line that is naturally characterized by a loss-of-function mutation of the T synthase chaperone protein Cosmc resulting in defective O-glycosylation (Ju T et al., Proc Natl Acad Sci USA. 2002 Dec. 24; 99(26):16613-8; FIG. 1a). To restore O-glycosylation and provide the antigen for CAR-T cells, Jurkat cells were transduced with lentiviral vectors carrying Cosmc and CD44v6 together with a selection marker (FIG. 1b). Strikingly, CD44v6 CAR-T cells recognized and killed more efficiently O-glycosylation incompetent Jurkat cells (44v6pos/Cosmcneg) than O-glycosylation competent Jurkat cells (44v6pos/Cosmcpos) (FIG. 1c). Accordingly, once transduced with the CD44v6 gene, naturally O-glycosylation competent K562 cells were lysed similarly to engineered glycosylation-competent Jurkat cells. These results indicate that O-glycosylation may hamper target cell killing by CAR-T cells.

To verify the impact of N-glycosylation, the inventors generated N-glycosylation-defective pancreatic tumour cells, e.g. T3M4, by knocking-out the expression of the glycosyltransferase Mgat5 using the CRISPR-Cas9 technology (FIG. 2a). Inhibition on N-glycosylation was confirmed by decreased binding to the PHA-L (FIG. 2b), a lectin that specifically binds to Mgat5-modified branched N-glycans. Importantly, glycosylation inhibition did not significantly interfere with CD44v6 exposure on the cell membrane, which is pre-requisite for CAR targeting (FIG. 2c). Strikingly, hampering N-glycosylation on T3M4 cells dramatically improved their elimination by CD44v6 CAR-T cells (FIG. 2d). This effect is associated with improved CAR-T cell activation (FIG. 2e) and cytokine release (FIG. 21), suggesting more proficient antigen engagement. These findings were confirmed using the N-glycosylation inhibitor tunicamycin. To verify if improved antitumor activity resulted from a different CAR-induced intracellular signalling event, the inventors exploited “triple parameter reporter” (TPR) Jurkat T cells (Jutz S et al., J Immunol Methods. 2016 March; 430:10-20 and FIG. 3a), in which different fluorescent proteins are placed under specific control of transcription factors (TFs) turned on during T-cell activation, e.g. NFAT, NF-kB and AP-1. These cells were transduced with the CD44v6 CAR and stimulated with different target cells. TFs elevation was stronger in the case of N-glycosylation defective T3M4 cells (FIG. 3b, left). As expected, no activation was observed in the case of TPR Jurkat cells transduced with the CD19 CAR and stimulated with CD19-negative target cells (FIG. 3b, right).

Altogether, these results indicate that glycosylation inhibits target cell elimination by CAR-T cells, possibly by sterically interfering with antigen recognition. Considering that solid tumours are characterized by several glycosylation alterations (Pinho S S et al., Nat Rev Cancer. 2015 September; 15(9):540-55), especially including an increased branching of N-glycans, pharmacological interference with the generation of such cumbersome sugar structures was assessed to verify if it might increase tumour cell recognition and killing by CAR-T cells.

### Example 2: 2DG Blocks N-Glycosylation on Pancreatic Cancer Cells

Searching for glycosylation inhibitors to safely use in combination with CAR-T cells, the inventors focused their attention on 2-Deoxy-D-Glucose (2DG). 2DG is a potent inhibitor of both glycolysis and N-linked glycosylation, which proved good tolerability in humans possibly thanks to its preferential accumulation in tumour cells, compared to healthy cells, as a consequence of the Warburg effect (Singh D et al., Strahlenther Onkol. 2005 August; 181(8):507-14; Stein M et al., Prostate. 2010 Sep. 15; 70(13):1388-94; Raez L E et al., Cancer Chemother Pharmacol. 2013 February; 71(2):523-30; Magistroni R et al., J Nephrol. 2017 August; 30(4):511-519; Xi H et al., IUBMB Life. 2014 February; 66(2):110-21). For these reasons, the inventors started investigating if 2DG might increase antitumor activity of CAR-T cells against solid tumours. As a first tumour model they used pancreatic adenocarcinoma, a cancer for which the development of new therapeutic options is particularly urgent (only the 5% of people are alive 5 years after diagnosis, World Cancer Report 2014 WHO).

Similarly to what happens after knocking out the Mgat5 gene, 2DG was able to potently inhibit N-glycosylation, as indicated by reduced binding to PHA-L (FIG. 4a) and by molecular-weight shift of beta-1 integrin, a model protein used for western blot analysis (FIG. 4b). This effect was already detectable after 5 hours (FIG. 4c) and lasted 24 hours after washing out 2DG (FIG. 4d). Moreover, it was already present when using low doses of 2DG (FIG. 4e), which were unable to inhibit glycolysis (FIG. 4f), suggesting that the selective blockade of N-glycosylation with 2DG is feasible. To note, low doses of 2DG had no impact on tumour cell viability (FIG. 4g) and proliferation (FIG. 4h), suggesting poor efficacy of 2DG as monotherapy.

Since expression on the cell surface is a prerequisite for CAR targeting, the inventors sought to examine if de-glycosylated antigens generated upon treatment with 2DG were able to reach the cell membrane. To this aim, they performed biotin-enrichment western blot analysis (see methods). Importantly, de-glycosylated forms of beta-1 integrin and CD44v6 were clearly detected among surface proteins upon treatment with 2DG (FIG. 5a). Treatment with the PNGase enzyme, which is able to cut glycans on mature proteins, was used as control and produced similar results. Surface expression levels of beta-1 integrin and CD44v6 measured by FACS were maintained as well (FIG. 5b).

Altogether these results indicate that low doses of 2DG are not cytotoxic per se for tumour cells but can inhibit N-glycosylation without interfering with proteins exposure on the cell surface.

### Example 3: 2DG Increases Killing of Pancreatic Cancer Cells by CD44v6 CAR-T Cells

After proving that 2DG induces the exposure of de-glycosylated antigens, the inventors investigated the antitumor activity of a combined approach based on 2DG plus CAR-T cells.

To avoid the potentially confusing activity of 2DG on T cells, tumour cells were pre-treated with 2DG before being co-cultured with CAR-T cells in the absence of 2DG. While expressing CD44v6, both PT45 and T3M4 were poorly targeted by CD44v6 CAR-T cells alone (FIG. 6a-b, red bars and lines). Strikingly, however, pre-treatment with 2DG sensitized tumour cells to recognition by CD44v6 CAR-T cells, significantly increasing their elimination (FIG. 6a-b, blue bars and lines). Notably, this effect was not simply additive but synergic, since it was above what could be expected from the individual antitumor activity of 2DG (which was lacking) and CAR-T cells alone (which was minimal)

By taking advantage of CAR-transduced Jurkat TPR cells, the inventors demonstrated that improved tumour cell killing also associated with improved T-cell activation (FIG. 6c), as was the case of Mgat5 knocked-out cells (see EXAMPLE 1).

### Example 4: 2DG does not Increase the Killing of Healthy Keratinocytes by CD44v6 CAR T Cells

To shed some light on the safety profile of the approach, the inventors started by evaluating the effect of 2DG alone towards healthy peripheral blood mononuclear cells (PBMCs). The same doses of 2DG able to inhibit tumour glycosylation failed to interfere with PBMCs glycosylation (FIG. 7a) and did not impact on the viability of CD3+ T cells, CD19+ B cells, CD14+ monocytes and CD15+ granulocytes (FIG. 7b).

Next, the inventors investigated the effect of the combined approach on healthy cells that can be potentially targeted by CAR-T cells. They previously reported both in vitro (Casucci M et al., Blood. 2013 Nov. 14; 122(20):3392-4) and in vivo that human keratinocytes, while expressing CD44v6, are not targeted by CD44v6 CAR-T cells. To verify if 2DG might increase their killing, human primary keratinocytes were exposed to 2DG before being co-cultured with CD44v6 CAR-T cells. Importantly, the same dose of 2DG able to enhance tumour cell recognition failed to inhibit keratinocyte glycosylation (FIG. 7c) and to increase keratinocyte elimination by CD44v6 CAR-T cells (FIG. 7d). These data support that glycosylation inhibitors improve the efficacy of CAR-T cell therapy. In particular thanks to the Warburg effect, 2DG improves the efficacy of CD44v6 CAR-T cell therapy without increasing toxicity against healthy tissues.

### Example 5: Treatment with 2DG Sensitizes Pancreatic Tumour Cells to CD44v6 CAR-T Cell Therapy In Vivo

The inventors next sought to evaluate if glycosylation inhibitors, such as 2DG, can sensitize tumour cells to killing by CAR-T cells in a pancreatic adenocarcinoma xenograft mouse model. To this aim, they used two different settings, i e minimal residual-disease (MRD) with a high CAR-T cell dose (FIG. 8a) and high tumour burden (HTB) with low CAR-T cell dose (FIG. 8b) in order to test the combinatorial treatment modality in both a more permissive setting as well as in a more challenging one, respectively. Briefly, NSG mice were injected with 44v6pos/19neg T3M4 cells expressing a secreted luciferase that allows the easy monitoring of tumour growth by simply analysing blood samples (Falcone L et al., Methods Mol Biol. 2016; 1393:105-11). After 2 or 7 days (MRD and HTB settings, respectively) mice received 2DG and were treated with different doses of CD44v6 or CD19 CAR-T cells (see Methods). Tumour growth was weekly monitored through bioluminescent analysis of blood samples. Whereas in the MRD setting, CD44v6 CAR-T cells were able alone to clear pancreatic tumour cells (FIG. 8c), in the HTB setting mice receiving CD44v6 CAR-T cells significantly benefited from 2DG administration (FIG. 8d). Interestingly, at sacrifice, tumour-infiltrating CD44v6 CAR-T cells from mice that received 2DG included a significantly lower frequency of cells expressing exhaustion and senescence markers compared to mice that did not receive 2DG, suggesting a better anti-tumour activity in the long-term (FIG. 8e). Altogether, these results showed efficacy of the combined treatment against a very aggressive pancreatic cancer cell line even in the setting where CAR-T cell alone were uncapable to mediate any antitumor activity.

### Example 6: The Combined Approach is Feasible with Different CAR Specificities

To verify if the synergistic effect between 2DG and CAR-T cells is common to other CAR specificities, the inventors took advantage of CEA CAR-T cells. The inventors chose CEA because it is a heavily glycosylated protein (60% of its weight comes from carbohydrates) over-expressed on a wide variety of solid tumours. Recently, CEA CAR-T cells proved hint of efficacy in the absence of significant toxicities in patients with liver metastasis of pancreatic cancer or colorectal carcinoma (Katz S C et al., Clin Cancer Res. 2015 Jul. 15; 21(14):3149-59; Zhang C et al., Mol Ther. May 3; 25(5):1248-1258), indicating that strategies to increase antitumor efficacy of CEA CAR-T cells are of great interest in the field.

In the present invention, it is demonstrated that, similarly to what observed with CD44v6 CAR-T cells (EXAMPLE 1), CEA CAR-T cells kill more efficiently N-glycosylation defective T3M4 cells than N-glycosylation competent cells (FIG. 9a). Also in this case, increased tumour killing was accompanied by increase T-cell activation, as indicated by analysing CD69 upregulation (FIG. 9b) and by exploiting Jurkat TPR cells as described in EXAMPLE 1 (FIG. 9c).

Most importantly, while pre-treatment with 2DG did not increase recognition of CEAneg PT45 tumour cells by CEA CAR-T cells (FIG. 9d), it significantly improved the elimination of CEApos BxPC3 and T3M4 tumour cells (FIG. 9e-f). Again, such improvement associated with increased CAR-T cell activation (FIG. 9g-h) and cytokine release (FIG. 9i).

Altogether, these results support the application of this combined strategy with different CAR specificities.

### Example 7: The Synergistic Effect is Applicable to Different Tumour Types

To verify if the synergistic effect between glycosylation inhibitors (such as 2DG) and CAR-T cells can be exploited with different tumour types, the inventors screened different solid (FIG. 10a) and hematologic (FIG. 11a) tumour cell lines for their glycosylation status, by exploiting PHA-L staining. Albeit expected variability, several cell cancer lines both from solid and hematopoietic types were found highly N-glycosylated, indicating that different cancer types, including pancreatic, lung and bladder solid tumours as well as AML, ALL and MM, can be targeted with the approach that combines glycosylation inhibitors and CAR T-cell treatments.

To functionally prove it, the inventors performed co-culture assays with some of the cell lines analysed. Strikingly, a negative correlation was observed between N-glycosylation levels and killing by CD44v6 CAR-T cells (FIG. 10b), further supporting that glycosylation negatively impacts CAR-T cell recognition. Moreover, the inventors proved that inhibiting glycosylation, in particular N-glycosylation with an inhibitor, in particular 2DG, drastically increase the elimination of highly glycosylated tumours that are barely targeted by CAR-T cells alone (FIG. 10c).

Altogether, these results support the application of this combined strategy for the treatment of multiple tumour types.

1. A method for the treatment and/or prevention of cancer, comprising administering a glycosylation inhibitor and a CAR cell therapy to a patient in need thereof.

2. The method according to claim 1 wherein said at least ono glycosylation inhibitor improves the therapeutic potential of said CAR cell therapy and/or improves CAR cell activation and/or increases antigen engagement and/or sensitizes tumour cells to recognition by the CAR cell therapy and/or increases elimination of tumour cells.

3. A method for increasing tumour cell killing, comprising exposing a tumour cell to a CAR cell therapy and a glycosylation inhibitor.

4. The method according to claim 1 wherein the CAR cell therapy is a CAR-T cell therapy or a CAR-NK cell therapy, and the T cell is optionally an autologous or allogeneic T cell.

5. The method according to claim 1 wherein said glycosylation inhibitor is selected from the group consisting of: a O-glycosylation inhibitor, a N-glycosylation inhibitor, a P-glycosylation inhibitor, a C-glycosylation inhibitor, a S-glycosylation inhibitor or a combination thereof.

6. The method according to claim 1 wherein said glycosylation inhibitor is selected from the group consisting of: a mannose analog, 2-deoxyglucose, 3-deoxy-3-fluoroglucosamine, 4-deoxy-4-fluoroglucosamine, 2-deoxy-2-fluoro-glucose, 2-deoxy-2-fluoro-mannose, 6-deoxy-6-fluoro-N-acetylglucosamine, 2-deoxy-2-fluorofucose, and 3-fluoro sialic acid tunicamycin, castanospermine, australine, deoxynojirimycin, swainsonine, deoxymannojirimycin, kifunensin, mannostatin, neuraminidase inhibitors, inhibitors of glycosyltransferases.

7. The method according to claim 1 further comprising a therapeutic agent, wherein said therapeutic agent is optionally an antibody, or a checkpoint inhibitor antibody.

8. The method according to claim 1 wherein the glycosylation inhibitor is 2-deoxyglucose.

9. The method according to claim 1 wherein the cancer is a solid or haematopoietic or lymphoid tumor.

10. The method according to claim 9, wherein the solid tumor is selected from the group consisting of: colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angio genesis, spinal axis tumor, brain stem glioma, pituitary adenoma, Kaposi's sarcoma, epidermoid cancer, squamous cell cancer, environmentally induced cancers, combinations of said cancers, and metastatic lesions of said cancers.

11. The method according to claim 9, wherein the haematopoietic or lymphoid tumor is selected from the group consisting of: chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia, combinations of said cancers, and metastatic lesions of said cancers.

12. The method according to claim 1 wherein the glycosylation inhibitor is administered prior to the CAR cell therapy.

13. The method according to claim 1 wherein the glycosylation inhibitor is administered concomitantly to the CAR-T cell therapy.

14. (canceled)

15. (canceled)

16. An isolated population or subpopulation of CAR cells or an isolated CAR cell that is contacted with at least one glycosylation inhibitor.

17. The isolated population or subpopulation of CAR cells or the isolated CAR cell of claim 16, wherein the isolated population or subpopulation of CAR cells or the isolated CAR cell are CAR-T cells or an isolated CAR-T cell.
